(19) United States
(12) Patent Application Publication
(10) Pub. No.: US 2013/0011413 A1
(43) Pub. Date: Jan. 10, 2013

(54) Method and Pharmaceutical Composition for Treatment of Intestinal Disease

(75) Inventors: Yoichiro Iwakura, Tokyo (JP); Shigeru Kakuta, Tokyo (JP); Shunsuke Suzuki, Tokyo (JP)

(73) Assignee: The University of Tokyo

(21) Appl. No.: 13/575,949

(22) PCT Filed: Feb. 2, 2011
(86) PCT No.: PCT/JP2011/052156

§ 371 Date: Sep. 21, 2012

Related U.S. Application Data
(60) Provisional application No. 61/300,962, filed on Feb. 3, 2010.

(51) Int. Cl. A61K 39/395; A61P 1/00; A61P 35/00; C07K 16/24

(52) U.S. Cl. 424/158.1; 530/389.2

(57) ABSTRACT

Provided is a pharmaceutical composition for the treatment of intestinal disease which contains an inhibitor of inflammatory cytokine IL-1 family molecules and IL-17 family molecules, and in particular contains an IL-17F inhibitor. Also provided is a pharmaceutical composition for the treatment of intestinal disease which contains an IL-17F inhibitor and an IL-17A (IL-17) inhibitor. Inflammatory cytokine IL-1 family molecules and IL-17 family molecules promote tumorigenesis during onset of colorectal cancer, and it has been found that tumorigenesis can be suppressed by suppressing these cytokines.

### TECHNICAL FIELD

This invention relates to the use of interleukin (also referred to as “IL” in this specification)-related substances to treat intestinal disease. More specifically, it relates to the use of IL-related substances to suppress or inhibit the advance of colon polyps or colorectal cancer. In particular, it relates to the use of an IL-17F inhibitor typified by an anti-IL-17F antibody to suppress or inhibit the advance of colon polyps or colorectal cancer. This invention also relates to a pharmaceutical composition to be used to treat these conditions.

### BACKGROUND ART

### Inflammatory Cytokines

The process of malignant transformation, which involves cancer cell proliferation, infiltration, metastasis, and the like, can be regarded as being determined by the properties of the cancer cells themselves, but in fact the cancer cells and the surrounding environment are deeply involved. A cancer growing in the body is formed not only by cancer cells, but by the interactions with various cells that create an environment favorable to the growth of the cancer cells themselves (Non-patent Reference 1). Many of these are stromal cells, for example, neutrophils, eosinophils, macrophages, dendritic cells, and other such inflammatory cells that migrate from the bone marrow and peripheral blood, vascular cells, epithelial cells, and fibroblasts. These relationships between the cancer environment and inflammatory cytokines have drawn attention in recent years.

Cytokines are divided into inflammatory cytokines (IL-1, IL-6, IL-8, IL-17, IFNγ, G-CSF, and the like) and anti-inflammatory cytokines (IL-4, IL-10, IL-11, IL-13, TGFβ, and the like), and the type of inflammation is decided by the immune cells that are activated. For example, if mainly IFNγ is produced, Th1 type inflammation occurs; if IL-4 is produced, Th2 type inflammation occurs (Non-patent References 2-5). Thus, there are conflicting reports to the effect that inflammatory cytokines suppress tumors since they control mechanisms that activate immune cells and cytotoxic T cells and remove foreign bodies and that the inflammatory milieu created by inflammatory cytokines promotes tumors (Non-patent References 6-8).

The tumor-suppressing effect of inflammatory cytokines depends on activation of the immune system. The immune system maintains the homeostasis of the organism by recognizing and eliminating not only bacteria, viruses, and other such extrinsic substances that invade from outside the body but also intrinsic foreign bodies that develop within the body. The establishment of natural immunity, which acts by recognizing common characteristics of many pathogens and distinguishing whether they are self or non-self, and adaptive immunity, which recognizes a wide range of pathogens, is indispensable to such mechanisms. CD4+T cells are known to be responsible for control of immune mechanisms in adaptive immunity. CD4+T cells differentiate into three representative subsets, Th1 cells, Th2 cells, and Th17 cells, through the interaction of naïve T cells with antigen in the peripheral lymph nodes (Non-patent References 5 and 9). The CD4+T cells differentiated into the respective subsets continue to proliferate cooperatively with each other or exclusively and regulate activation of the immune system. Th1 cells activate CD8+T cells, NK cells, and the like through the production of IFNγ, an inflammatory cytokine, and these activated cells bear the responsibility of protecting the organism against intracellular parasitic infection. Activated CD8+T cells also act as a mechanism to eliminate tumor cells, which are intrinsic foreign bodies produced by mutation of autologous cells (Non-patent Reference 10).

Activation of tumor immunity by inflammatory cytokines such as IFN-γ was proven by experiments using mice (Non-patent References 11 and 12). IFN-γ not only activates immune cells but also acts on tumor cells themselves and is known to have a direct growth-suppressing effect at the same time as promoting the expression of MHC classes I and II. Such an antitumor effect by cytotoxic T cells is useful in highly antigenic malignant melanoma and the like, but, unlike bacteria and such, tumor cells rarely have antigens that differ clearly from those of the host. It is difficult to say that tumor immunity works effectively in the body since tumor cells not only are weakly antigenic but tumor cells themselves produce TGF-β and IL-10 which attenuate the immune response (Non-parent Reference 13). The extent to which tumor immunity works in the course of carcinogenesis in the intestine in particular is not known.

On the other hand, the inflammatory milieu created by inflammatory cytokines is also reported to promote tumorigenesis (Non-patent Reference 6). Carcinogenesis is a disease based on genomic aberrations, as observed in familial tumors. Inflammatory cells made to migrate by inflammatory cytokines produce active oxygen, and this active oxygen is known to be deeply involved in carcinogenesis as it triggers DNA mutation, DNA cleavage, base modification, and other such direct DNA damage. There are also many reports on inflammatory conditions and promotion of tumorigenesis, such as reports that inflammatory cytokines enhance VEGFA and other such angiogenic factors and promote cell proliferation and metastasis by inducing angiogenesis in the tumor milieu (Non-patent References 15-17). Inflammation due to bacterial infection is also known to be a risk factor for carcinogenesis, as in Schistosoma japonicum being a risk factor for colorectal cancer, hepatitis C virus for liver cancer, and Helicobacter pylori for stomach cancer. However, since inflammatory cytokines also act to protect against these infections (Non-patent Reference 5), the relationship between inflammatory cytokines and carcinogenesis is complex. In the intestine in particular, many enteric bacteria are resident, and the role of inflammatory cytokines in the pathogenesis of colorectal cancer is even harder to predict since bacteria that induce inflammation are also present among these enteric bacteria, depending on changes in the flora.

### IL-1 Family Molecules

IL-1 family molecules are produced from macrophages and various other immune cells and play an important role in rheumatoid arthritis and other such inflammatory diseases (Non-patent References 18-22). They also control the expression of cyclooxygenase (COX) 2 downstream. COX2 is a rate-determining enzyme in the metabolism of prostaglandin (PG) H2 to PGG2. PGG2 is metabolized into PGE2, angiogenesis and apoptosis inhibition occur, and tumorigenesis is promoted. COX2 thus plays a very important role in the onset of colorectal cancer and stomach cancer. It is understood from the analysis of multiple mutant mice of colorectal cancer model mice and COX2 knockout mice that tumorigenesis is dramatically suppressed in mice that do not produce COX2 (Non-patent Reference 23). Immunologically as well, the risk of developing colorectal cancer is known to be suppressed in habitual users of COX1 and COX2 inhibitors (aspirin) (Non-patent Reference 24).

### IL-17 Family Molecules

The signal of above IL-1 is also known to be responsible for Th17 differentiation regulation downstream (Non-patent Reference 25). In particular, IL-17 (also commonly referred to as “IL-17A;” the terms “IL-17” and “IL-17A” are used synonymously in this specification as well) is produced from Th17 cells and is an important factor in inflammatory diseases such as rheumatoid arthritis and multiple sclerosis. Expression of IL-17A is found to be heightened in these inflammatory diseases. Analyses of knockout mice show it to be very important in the development of collagen-induced arthritis and experimental autoimmune spondylitis, and it has also been demonstrated to participate in defense mechanisms against bacterial and protozoal infection (Non-patent Reference 26).

On the other hand, “IL-17F” has the highest homology with IL-17A of the six IL-17 family molecules. Although they are said to bind to the same receptors (Non-patent References 27-29), IL-17A is produced from T cells while IL-17F is also produced outside T cells, and its effects are also known not to match those of IL-17A in the immune system (Non-patent Reference 26). In addition, IL-17A plays an important role in the onset of inflammatory autoimmune diseases, as was mentioned above, but analyses of knockout mice have clarified that IL-17F virtually does not participate (Non-patent Reference 26).

However, IL-17F was found to participate in opportunistic infections in mucosal tissue, according to a report by Ishigame et al. In sum, while abscesses formed due to growth of Staphylococcus aureus, an opportunistic pathogen, beneath the skin of the nose as IL17A/F knockout mice aged, no infection occurred even with aging in IL-17A or IL-17F alone knockout mice, showing that IL-17A and IL-17F play equally important roles in protection against infection (Non-patent Reference 26). Similarly, IL-17A, IL-17F, and IL-17A/F knockout mice were more susceptible to colonic bacterial infection than the wild type in the results of an infection experiment by Citrobacter rodentium, a pathogenic colon bacterium of mice (Non-patent Reference 26). IL-17 family molecules are intimately related to variations in intestinal flora and associated inflammations and are also important to maintaining the homeostasis of the intestine.

Thus, since IL-17 family molecules are important inflammation factors, while they are also involved in maintaining the homeostasis of the intestinal flora, it is still difficult to predict the relationship between intestinal cancer and IL-17 family molecules.

### Apc^{Min/+} Mice

Apc^{Min/+} mice were used as a colorectal cancer model mice in this research. Apc is known as a typical tumor suppressor gene of colorectal cancer and acts in the body to control β-catenin, a nuclear transcription factor. Virtually no β-catenin is present in the nucleus since β-catenin is trapped by APC, and the trapped β-catenin is phosphorylated, ubiquitinated, and degraded by proteasome (Non-patent References 30-32). However, if a mutation occurs in the Apc gene and it loses this function, β-catenin is not phosphorylated and, as a result, not degraded, allowing it to migrate to the nucleus and act as a transcription factor. Mutation of Apc serves is an early stage of cancer since the transcription products include cyclin D and other such factors involved in cell proliferation (Non-patent References 33-35). Since hemi-allele loss called as loss of heterozygosity (LOH) occurs frequently in the colon in particular, colorectal cancer develops with age if there is even one mutation of the Apc gene. Approximately 80% of colorectal cancer patients are known to have mutations of this Apc gene (Non-patent Reference 36). Apc^{Min/+} mice having a nonsense point mutation in the region that encodes the Apc gene therefore are model mice of familial adenomatous polyposis in which polyps develop spontaneously throughout the intestine with age. Model mice also used in the working examples of this specification were multiple mutant mice produced by crossing the above Apc^{Min/+} mice with Il1rn^{-/-} mice (refer to Non-patent Reference 37), Il17a^{-/-} (refer to Non-patent Reference 38), Il17f^{-/-}, Il17a/f^{-/-} mice (refer to Non-patent Reference 26 and the document Supplemental Data; http://www.immunity.com/supplemental/S1074-7613(08)00554-2).

### Participation of IL-17 Family Molecules in the Pathogenesis of Colorectal Cancer

Contradictory reports have appeared up to now regarding IL-17 and carcinogenesis (Non-patent Reference 40). Notably, there are almost no reports that implicate IL-17 in the pathogenesis of intestinal cancer in humans and mice. The group of Cynthia L. Sears et al. recently established a system that induces colorectal cancer in a very short time in Apc^{Min/+} mice by transplanting and establishing enterotoxigenic Bacteroides fragilis (ETBF), a type of enteric bacterium, to cause the cells to produce toxin and to induce chronic inflammation. Since carcinogenesis is suppressed by anti-IL-17A antibody in this system, Th17 and IL17A produced therefrom were reported to be important to the promotion of carcinogenesis (Non-patent Reference 41). Anti-human IL-17 (IL-17A) antibody known to antagonize IL-17A has been reported (Patent Reference 1).

Nonetheless, there are still no reports that suggest a relationship between cancer and IL-17F, which is known to be unimportant in autoimmune diseases. Therefore, mechanisms of action of IL-17F during carcinogenesis and the relationship between spontaneously occurring intestinal cancer and these cytokines have not been explored.

### Colorectal Cancer Suppression

In 2004, Dunn, G. P. et al. reported based on a carcinogenesis experiment using immunodeficient mice that the immune system protects the body from cancer and drew attention to the antitumor immune repose by cytotoxic T cells (Non-patent Reference 42). Tumor promotion was seen and the infiltration of CD8+T cells is known to be suppressed as a result of experiments that transplanted B16 melanoma to IL-17 (IL-17A) knockout mice (Non-patent Reference 43). Activation of CTL by IL-17A also appeared based on these findings to be effective in highly antigenic cancers. It was also reported after the priority date of this application that the intestinal polyp formation of Apc^{Min/+} mice is suppressed in IL-17A knockout mice and can also be suppressed by administration of anti-IL-17A antibody (Non-patent Reference 44).

Antibody drugs that target angiogenic factors have already been found to be effective. A phase III clinical study of anti-human VEGFA neutralizing antibody (Avastin) was conducted in colorectal cancer patients and demonstrated a remarkable life-prolonging effect (Non-patent Reference 45). However, angiogenesis inhibitors are not a panacea and are also reported to have serious adverse effects such as hypertension, kidney disorders, and thrombus formation. Specific inhibition of angiogenesis factors expressed at high levels in the cancer cell locale in epithelial cells of the colon and other parts of the intestine is therefore a very interesting topic.

There is also, for example, among the CD4+T cell subsets a cell population called regulatory T cells (Treg) that produce IL-10 to suppress inflammation. It was understood based on the 2009 experiments of Khashayarsha Khazaie et al. that tumorigenesis is suppressed as a result of transplanting Treg to colorectal cancer model mice. However, surprisingly enough, despite the large number of Treg that infiltrated the cancer cell locale, the Treg that infiltrated produced IL-17A, without producing IL-10 which is an anti-inflammatory cytokine. The transplanted Treg were also understood to be IL-10-producing Treg soon after transplant but to change into IL-17A-producing Treg over time (Non-patent Reference 46). It is not understood, however, how the IL-17A produced from these IL-17A-producing Treg works within the body.

The relationship between the intestinal flora and colorectal cancer is also important. The enteric bacterium ETBF used by the above-mentioned group of Cynthia L. Sears et al. has drawn attention because it is present in many colorectal cancer patients and causes colitis, especially when infection occurs in early childhood (Non-patent Reference 41). The experimental results of Ruslan Medzhitov et al. also demonstrated that tumorigenesis is suppressed in mice with the signal adaptor molecule Myd88, downstream of TLR, a sensor molecule of stimulation by enteric bacteria, knocked out (Non-patent Reference 47). It was also understood that there are bacteria that promote IL-17 production, given the different types of bacteria resident in the intestinal flora depending on differences in the rearing environment. Experiments by Dan R. Littman et al. in 2009 demonstrated that the number of Th17 cells present in the intestine differed in C57BL/6J mice raised at Jackson Co. and C57BL/6J mice raised at Taconic Co., that the cause of this was enteric bacteria called segmented filamentous bacteria resident in the Taconic Co. mice, and that these bacteria promote IL-17 production (Non-patent Reference 48). It would be expected based on these results that IL-17 family molecules are produced by stimulation of enteric bacteria and that homeostasis of the intestinal flora is maintained by the IL-17 family molecules produced.

### PRIOR ARTS REFERENCES

### Non-Patent References

Non-patent Reference 1: Margareta M. Mueller & Norbert E. Fusenig: Friends or foes—bipolar effects of the tumor stroma in cancer: Nature Reviews Cancer 4, 839-849 (2004)
Non-patent Reference 2: Ming O. Li, Shomyseh Sanjabil and Richard A. Flavell: Transforming Growth Factor-β Controls Development, Homeostasis, and Tolerance of T Cells by Regulatory T Cell-Dependent and -Independent Mechanisms: Immunity 25, 455-471 (2006)
Non-patent Reference 3: K W Moor, A O'Garra, R W Malefyt, P Vieira, and, T R Mosmann: Interleukin-10: Annual Review of Immunology, 11: 165-190 (1993)
Non-patent Reference 4: Laurie H. Glimcher & Kenneth M. Murphy: Lineage commitment in the immune system: the T helper lymphocyte grows up: Genes & Development: 14: 1693-1711 (2000)
Non-patent Reference 5: T R Mosmann, &, R L Coffman: TH1 and TH2 Cells: Different Patterns of Lymphokine Secretion Lead to Different Functional Properties; Annual Review of Immunology 7: 145-173 (1989)
Non-patent Reference 6: Murugaiyan, G., S. Martin, and B. Saha. 2007. CD40-induced countercurrent conduits for tumor escape or elimination? Trends Immunol. 28: (467-473)
Non-patent Reference 7: Zitvogel, L., A. Tesniere, and G. Kroemer.: Cancer despite immunosurveillance: immunoselection and immunosubversion. Nat. Rev. Immunol. 6 715-727 (2006)
Non-patent Reference 8: Murugaiyan, G., S. Basak, and B. Saha.: Reversal of tumor induced dendritic cell paralysis: a treatment regimen against cancer. Curr. Immunol. Rev. 2: 261-272 (2006)
Non-patent Reference 9: Carmen Infante-Duarte, Heidi F. Horton, Michael C. Byrne, and Thomas Kamradt: Microbial Lipopeptides Induce the Production of IL-17 in Th Cells: Journal of immunology: 165: 6107-6115 (2000)
Non-patent Reference 10: Laurence Zitvogel, Antoine Tesniere, Guido Kroemer: Cancer despite immunosurveillance: immunoselection and immunosubversion: Nature Reviews Immunology 6, 715-727 (2006)
Non-patent Reference 11: Hans Strander & Stefan Einhorn: Interferons and the tumor cell: Biotherapy 8: 213-218 (1996)
Non-patent Reference 12: Gavin P. Dunnl, Catherine M. Koebell & Robert D. Schreiber: Interferons, immunity and cancer immunoediting: Nature Reviews Immunology 6, 836-848 (2006)
Non-patent Reference 13: Alexander J. Mullen & Peggy A. Scherle: Targeting the mechanisms of tumoral immune tolerance with small-molecule inhibitors: Nature Reviews Cancer 6, 613-625 (2006)
Non-patent Reference 14: Numasaki, M., M. T. Lotze, and H. Sasaki.: Interleukin-17 augments tumor necrosis factor-α-induced elaboration of proangiogenic factors from fibroblasts. Immunol. Lett. 93: 39-43 (2004)
Non-patent Reference 15: Takahashi, H., M. Numasaki, M. T. Lotze, and H. Sasaki.: Interleukin-17 enhances bFGF-, HGF- and VEGF-induced growth of vascular endothelial cells. Immunol. Lett. 98: 189-193 (2005)
Non-patent Reference 16: Kehlen, A., K. Thiele, D. Riemann, N. Rainov, and J. Langner.: Interleukin-17 stimulates the expression of IκBα mRNA and the secretion of IL-6 and IL-8 in glioblastoma cell lines. J. Neuroimmunol. 101: 1-6. (2000)
Non-patent Reference 17: Kehlen, A., K. Thiele, D. Riemann, N. Rainov, and J. Langner.: Interleukin-17 stimulates the expression of I_B_ mRNA and the secretion of IL-6 and IL-8 in glioblastoma cell lines. J. Neuroimmunol. 101: 1-6. (2000)
Non-patent Reference 18: Dinarello, C. A.: Biologic basis for interleukin-1 in disease. Blood, 87, 2095-2147 (1996)
Non-patent Reference 19: Nakae, S., Horai, R., Komiyama, Y., Nambu, A., Asano, M., Nakane, A., and Iwakura, Y.: The role of IL-1 in the immune system. In “Cytokine Knockouts”, (ed. G. Fantuzzi), Human Press, Totowa, N.J., 95-109, (2003)
Non-patent Reference 20: Horai, R., Saijo, S., Tanioka, H., Nakae, S., Sudo, K., Okahara, A., Ikuse, T., Asano, M. and Iwakura, Y.: Development of chronic inflammatory arthropathy resembling rheumatoid arthritis in interleukin 1 receptor antagonist-deficient mice. J. Exp. Med, 191, 313-20 (2000)
Non-patent Reference 21: Saijo, S., Asano, M., Horai, R., Yamamoto, H., and Iwakura, Y.: Suppression of autoimmune arthritis in IL-1-deficient mice in which T cell activation is impaired due to low levels of CD40L and OX40 expression on T cells. Arth. Rheum., 46, 533-544 (2002)
Non-patent Reference 22: Nakae, S., Saijo, S., Horai, R., Sudo, K., Mori, S., and Iwakura, Y.: IL-17 production from activated T cells is required for the spontaneous development of destructive arthritis in mice deficient in IL-1 receptor antagonist. Proc. Natl. Acad. Sci. USA, 100, 5986-5990, (2003)
Non-patent Reference 23: Masanobu Oshima, Joseph E. Dinchuk, Stacia L. Kargman, Hiroko Oshima, Bruno Hancock, Elizabeth Kwong, James M. Trzaskos, Jilly F. Evans, ‡ and Makoto M. Taketo: Suppression of Intestinal Polyposis in ApcD716 Knockout Mice by Inhibition of Cyclooxygenase 2 (COX-2): Cell, 87, 803-809, (1996)
Non-patent Reference 24: Thun M J, Namboodiri M M, Heath S W Jr.: Aspirin use and reduced risk of fatal colorectal cancer. N Eng J Med 325: 1593-6 (1991)
Non-patent Reference 25: Yeonseok Chung, Seon Hee Chang, Gustavo J. Martinez, Xuexian O. Yang, Roza Nurieva, Hong Soon Kang, Li Ma, Stephanie S. Watowich, Anton M. Jetten, Qiang Tian and Chen Dong: Critical Regulation of Early Th17 Cell Differentiation by Interleukin-1 Signaling: Immunity 30 576-587 (2009)
Non-patent Reference 26: Ishigame, H., Kakuta, S., Nagai, T., Kadoki, M., Nambu, A., Komiyama, Y., Fujikado, N., Tanahashi, Y., Akitsu, A., Kotaki, H., Sudo, K., Nakae, S., Sasakawa, C., and Iwakura, Y.: Differential Roles of Interleukin-17A and -17F in Host Defense against Mucoepithelial Bacterial Infection and Allergic Responses. Immunity 30 108-119 (2009)
Non-patent Reference 27: Aggarwal S, Gurney A L. IL-17: prototype member of an emerging cytokine family. J Leukoc Biol 71 1-8 (2002)
Non-patent Reference 28: Kolls J K, Linden A. Interleukin-17 family members and inflammation. Immunity 21: 467-76 (2004)
Non-patent Reference 29: Weaver C T, Hatton R D, Mangan P R,: IL-17 Family Cytokines and the Expanding Diversity of Effector T Cell Lineages. Annu Rev Immunol 25: 821-52 (2007)
Non-patent Reference 30: Satoshi Ikeda, Shosei Kishida, Hideki Yamamoto, Hiroshi Murai, Shinya Koyama, Akira Kikuchi: Axin, a negative regulator of the Wnt signaling pathway, forms a complex with GSK-3β and β-catenin and promotes GSK-3β-dependent phosphorylation of β-catenin: The EMBO Journal 17, 1371-1384 (1998)
Non-patent Reference 31: Tsutomu Nakamura, Fumihiko Hamada, Takao Ishidate, Ken-ichi Anai, Kohichi Kawahara, Kumao Toyoshima, Tetsu Akiyama: Axin, an inhibitor of the Wnt signaling pathway, interacts with -catenin, GSK-3 and APC and reduced the -catenin level: Genes and cells 3, 395-403 (1998)
Non-patent Reference 32: Jurgen Behrens, Boris-Alexander Jerchow, Martin Wurtele, Jan Grimm, Christian Asbrand, Ralph Wirtz, Michael Kuhl, Doris Wedlich, and Walter Birchmeier: Functional Interaction of an Axin Homolog, Conductin, with -Catenin, APC, and GSK3: Science 283 596-599 (1998)
Non-patent Reference 33: B Rubinfeld, B Souza, I Albert, O Muller, S H Chamberlain, F R Masiarz, S Munemitsu, and P Polakis: Association of the APC gene product with beta-catenin: Science 262 1731-1734 (1993)
Non-patent Reference 34: LK Su, B Vogelstein, and KW Kinzler: Association of the APC tumor suppressor protein with catenins: Science 262 1734-1737 (1993)
Non-patent Reference 35: Ken M. Cadigan and Roel Nussel: Wnt signaling: a common theme in animal development: Genes Dev 11 3286-3305 (1997)
Non-patent Reference 36: Kenneth W. Kinzler and Bert Vogelstein: Lessons from Hereditary Colorectal Cancer: Cell 87 159-170 (1996)
Non-patent Reference 37: Horai, R., Asano, M., Sudo, K., Kanuka, H., Suzuki, M., Nishihara, M., Takahashi, M., and Iwakura, Y: Production of mice deficient in genes for IL-1α, IL-1β, IL-1α/β, and IL-1 receptor antagonist shows the IL-1β is crucial in turpentine-induced fever development and glucocorticoid induction. J. Exp. Med., 187: 1463-1475 (1998)
Non-patent Reference 38: Nakae, S., Komiyama, Y., Nambu, A., Sudo, K., Iwase, M., Homma, I., Sekikawa, K., Asano, M., and Iwakura, Y.: Antigen-specific T cell sensitization is impaired in IL-17-deficient mice, resulting in the suppression of allergic cellular and humoral responses. Immunity, 17, 375-387 (2002).
Non-patent Reference 39: http://www.broadinstitute.org/gsea/
Non-patent Reference 40: Gopal Murugaiyan and Bhaskar Saha: Protumor vs Antitumor Functions of IL-17: J. Immunol. 183; 4169-4175 (2009)
Non-patent Reference 41: Shaoguang Wu, Ki-Jong Rhee, Emilia Albesiano, Shervin Rabizadeh, Xinqun Wu, Hung-Rong Yen, David L Huso, Frederick L Brancati, Elizabeth Wick, Florencia McAllister, Franck Housseau, Drew M Pardoll & Cynthia L Sears: A human colonic commensal promotes colon tumorigenesis via activation of T helper type 17 T cell responses: Nature Medicin 15 1016-1022 (2009)
Non-patent Reference 42: Gavin P. Dunn, Lloyd J. Old, and Robert D. Schreiber: The Three Es of Cancer Immunoediting: Annual Review of Immunology 22: 329-360 (2004)
Non-patent Reference 43: Ilona Kryczek, Shuang Wei, Wojciech Szeliga, Linhua Vatan, and Weiping Zou: Endogenous IL-17 contributes to reduced tumor growth and metastasis: Blood 114 357-359 (2009)
Non-patent Reference 44: Chae W J, Gibson T F, Zelterman D, Hao L, Henegariu O, Bothwell A L.: Ablation of IL-17A abrogates progression of spontaneous intestinal tumorigenesis. Proc Natl Acad Sci USA. 107(12): 5540-4 (2010)
Non-patent Reference 45: Herbert Hurwitz, M.D., Louis Fehrenbacher, M.D., William Novotny, M.D., Thomas Cartwright, M.D., John Hainsworth, M.D., William Heim, M.D., Jordan Berlin, M.D., Ari Baron, M.D., Susan Griffing, B.S., Eric Holmgren, Ph.D., Napoleone Ferrara, M.D., Gwen Fyfe, M.D., Beth Rogers, B.S., Robert Ross, M.D., and Fairooz Kabbinavar, M.D: Endogenous IL-17 contributes to reduced tumor growth and metastasis: Blood: Bevacizumab plus Irinotecan, Fluorouracil, and Leucovorin for Metastatic Colorectal Cancer: New England Journal Of Medicine 350: 2335-2342 (2004)
Non-patent Reference 46: Elias Gounaris, Nichole R. Blatner, Kristen Dennis, Fay Magnusson, Michael F. Gurish, Terry B. Strom, Phillipp Beckhove, Fotini Gounari and Khashayarsha Khazaie: T-Regulatory Cells Shift from a Protective Anti-Inflammatory to a Cancer-Promoting Proinflammatory Phenotype in Polyposis: Cancer Res 69: 5490-5497 (2009)
Non-patent Reference 47: Seth Rakoff-Najoum and Ruslan: Medzhitov: Regulation of Spontaneous Intestinal Tumorigenesis Through the Adaptor Protein MyD88: Science 317 124-127 (2007)
Non-patent Reference 48: Ivaylo I. Ivanov, Koji Atarashi, Nicolas Manel, Eoin L. Brodie, Tatsuichiro Shima, Ulas Karaoz, Dongguang Wei, Katherine C. Goldfarb, Clark A. Santee, Susan V. Lynch, Takeshi Tanoue, Akemi Imaoka, Kikuji Itoh, Kiyoshi Takeda, Toshinori Umesaki, Kenya Honda, Dan R. Littman: Induction of Intestinal Th17 Cells by Segmented Filamentous Bacteria: Cell 139 485-498 (2009)

### Patent References

Patent Reference 1: International Publication WO2007/117749 pamphlet

### SUMMARY OF THE INVENTION

Thus, it cannot be said that the role of inflammatory cytokines in the pathogenesis of colorectal cancer has been fully explained. The present inventors therefore focused on the relationship between colorectal cancer and IL-1 family genes, which are important factors in inflammation (inflammatory cytokines). Specifically, the effects on tumorigenesis and inflammatory conditions caused by IL-1 were evaluated using mice in which knockout of the gene (Il1rn) of IL-1 receptor antagonist (RA), which acts as an endogenous antagonist of IL-1α,β, could be expected to make the IL-1 signal excessive and enhance expression of COX2.

In addition, as was mentioned above, IL-1 family molecules are known to act as regulatory factors of IL-17-producing T cells (Th17) downstream. IL-17 family molecules are thus also important factors in inflammation and, on the other hand, are also involved in maintaining the homeostasis of the intestinal flora, making it still difficult to easily predict the relationship between colorectal cancer and IL-17 family molecules. The present inventors therefore focused on the relationship between IL-17 family molecules and colorectal cancer, and evaluated whether these molecules act to promote or suppress tumorigenesis of colorectal cancer using mice having modified genes of IL-17 family molecules.

Specifically, multiple mutant mice were produced by crossing Apc^{Min/+} mice, which are model mice of familial adenomatous polyposis in which polyps develop spontaneously throughout the intestine with age, and mice deficient in IL-1 and IL-17 family genes (Il1rn^{-/-}, Il17a^{-/-}, Il17f^{-/-}, Il17a^{-/-}/f^{-/-}). The involvement of the inflammatory cytokines in polyp formation was investigated by comparing the size and number of polyps that developed in these mice and Apc^{Min/+} mice, and their mechanisms of action were clarified.

As a result, IL-1 family genes and IL-17 family molecules were demonstrated to be closely related to colorectal cancer.

In sum, both the number and size of polyps were shown to increase significantly in mice deficient in IL-1 receptor antagonist (Il1rn^{-/-}). As a result of comparing the polyps of Apc^{Min/+}-Il1rn^{-/-} multiple mutant mice and the polyps of Apc^{Min/+} mice, the expression of Il17a and Il17f was shown to be enhanced in the Apc^{Min/+}-Il1rn^{-/-} multiple mutant mice. Moreover, the number of polyps 3 mm or larger in size that developed in Apc^{Min/+}-Il17a^{-/-}/f^{+/-} mice was significantly decreased in comparison to Apc^{Min/+}-Il17a^{+/-}/f^{+/-} mice, and polyps 1 mm or larger in size decreased in Apc^{Min/+}-Il17a^{+/-}/f^{-/-} mice. The number of polyps that developed was decreased in Apc^{Min/+}-Il17a^{-/-}/f^{-/-} mice in comparison to the respective single knockout mice, and IL-17 and IL-17F were found to act as factors on fibroblasts to enhance angiogenesis. While not wishing to be bound by theory, it is suggested that IL-17 family molecules promote cell proliferation by enhancing angiogenesis and to promote tumorigenesis.

A comparison of the number of polyps that developed throughout the entire intestine in Apc^{Min/+}-Il17a^{-/-}/f^{+/-} mice and Apc^{Min/+}-Il17a^{+/-}/f^{-/-} mice demonstrated the number of polyps to be significantly decreased in Apc^{Min/+}-Il17a^{+/-}/f^{-/-} mice. It can be suggested as a result that, although IL-17 (IL-17A) is only produced by infiltrating cells in the polyp locale, IL-17F is produced by the intestinal epithelial cells themselves in addition to the infiltrating cells, making IL-17F production in the polyp locale greater than that of IL-17.

Based on the above results, it may be that IL-1 family molecules and IL-17 family molecules, especially the IL-17F molecule, can be newly added as targets of antibody therapy, which is expected to serve, along with surgical treatment, chemotherapy, radiation therapy, and immunotherapy, as a fifth method of treatment for tumors.

Therefore, in a first aspect of the present invention, a pharmaceutical composition for the treatment of intestinal disease containing an IL-17F inhibitor is provided.

Specifically, many previous studies have indicated that the effects of IL-17F are weaker than those of IL-17A. However, evidence was obtained that it is conceivable that the excessive production of IL-17F in the tumor locale plays a central role in tumorigenesis cells in the actual pathogenesis of colorectal cancer since IL-17F is produced from both epithelial cells and infiltrating. In the final analysis, this is reasonable to infer since a difference was seen even in the number of polyps 1 mm or larger in size that developed in Apc^{Min/+}-Il17a^{+/-}/f^{-/-} mice while no changes were seen in the number of polyps no larger than 3 mm in Apc^{Min/+}-Il17a^{-/-}/f^{+/-} mice, regardless of the fact that IL-17A and IL-17F act in the same way on fibroblasts and enhance angiogenesis. Based on the above findings, it is believed that inflammatory cytokine IL-1 family molecules and IL-17 family molecules act to promote tumorigenesis during onset of colorectal cancer, and that tumorigenesis can be suppressed by suppressing these cytokines, especially IL-17F.

The use and effects of an anti-IL-17F antibody as the above IL-17F inhibitor are illustrated in the working examples. Therefore, in the second aspect of the present invention, the above IL-17F inhibitor is an anti-IL-17F antibody, and a pharmaceutical composition for the treatment of intestinal disease containing an IL-17F inhibitor is provided.

In the third aspect of the present invention, a pharmaceutical composition for the treatment of intestinal disease using an IL-17A inhibitor in combination with an IL-17F inhibitor is provided. A typical IL-17A inhibitor is an anti-IL-17A antibody. The use and effects of a combination of anti-IL-17F antibody and anti-IL-17A antibody are also illustrated in the working examples.

In the fourth aspect of the present invention, a pharmaceutical composition for the treatment of intestinal disease is provided in which the intestinal disease to be treated by the IL-17F inhibitor is polyps or cancer in the intestine and the intestine is the large intestine. Therefore, advantageous embodiments of the present invention include pharmaceutical compositions to be used to prevent and/or treat colorectal cancer.

In the fifth aspect of the present invention, the use of an IL-17F mimetic, siRNA, and antisense RNA having IL-17F-inhibiting activity as another IL-17F inhibitor for the above purposes is also contemplated.

Thus, the present invention contemplates a method of treating intestinal disease, typically polyps or cancer in the intestine, more specifically colorectal cancer patients, using an IL-17F inhibitor. The present invention also intends the use of an IL-17F inhibitor to manufacture a pharmaceutical composition to treat intestinal disease, typically polyps or cancer in the intestine, more specifically colorectal cancer patients.

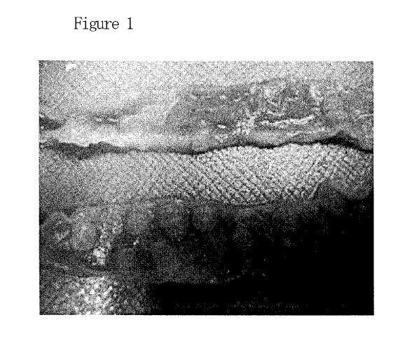

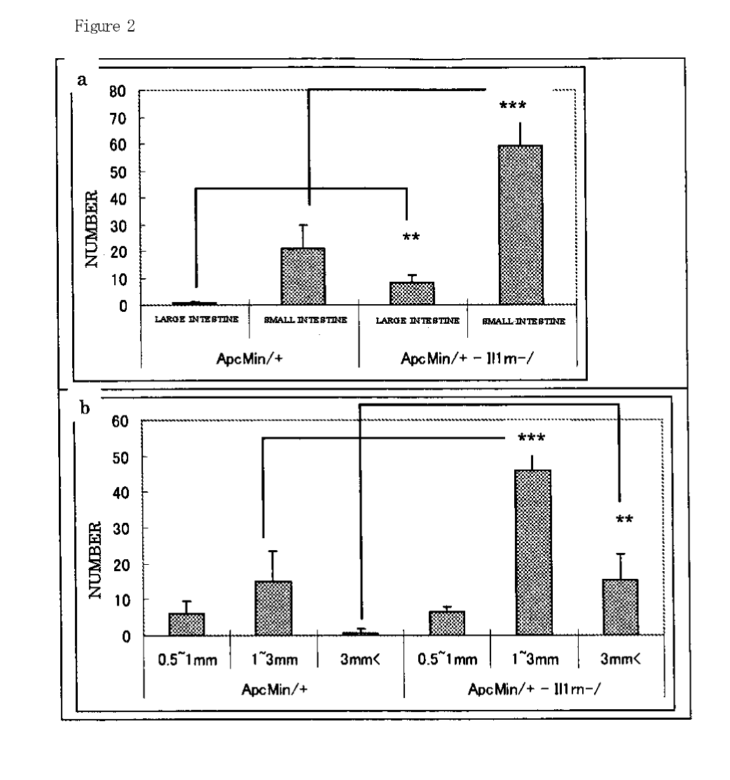

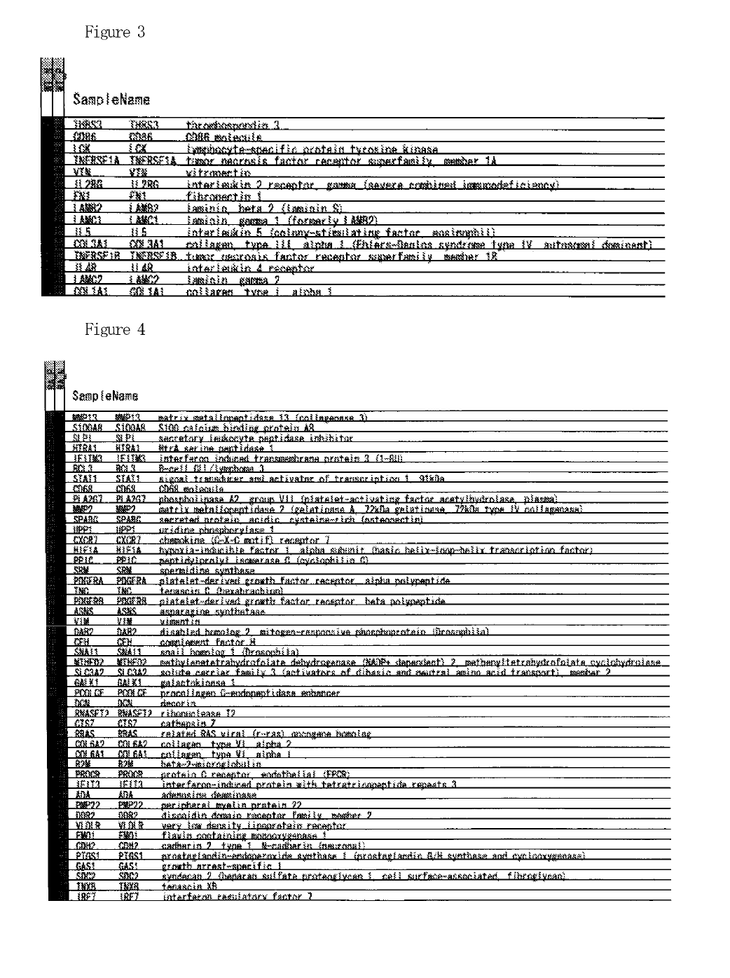

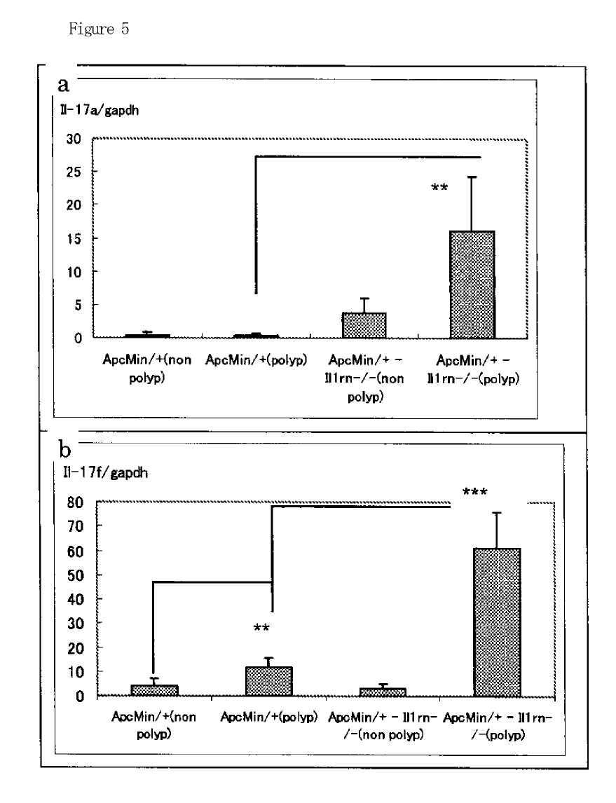

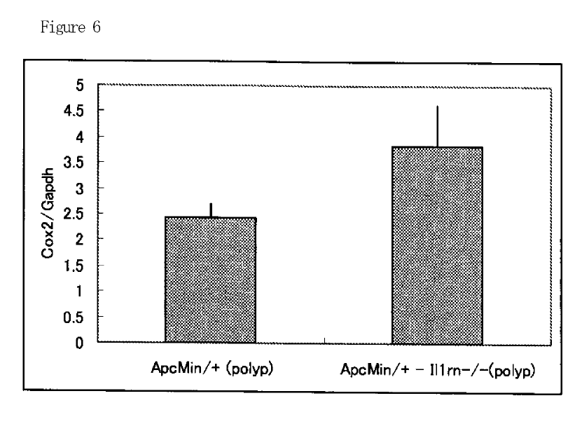

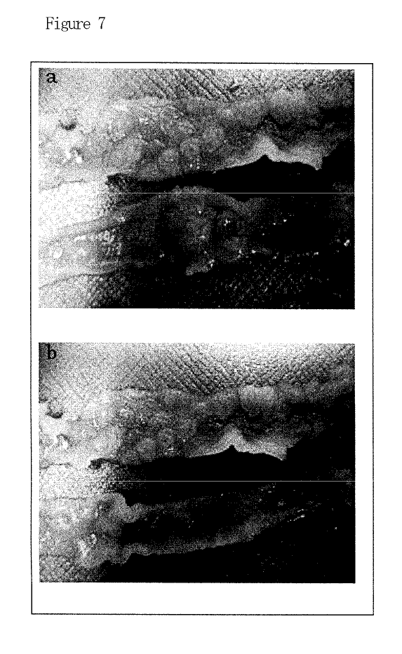

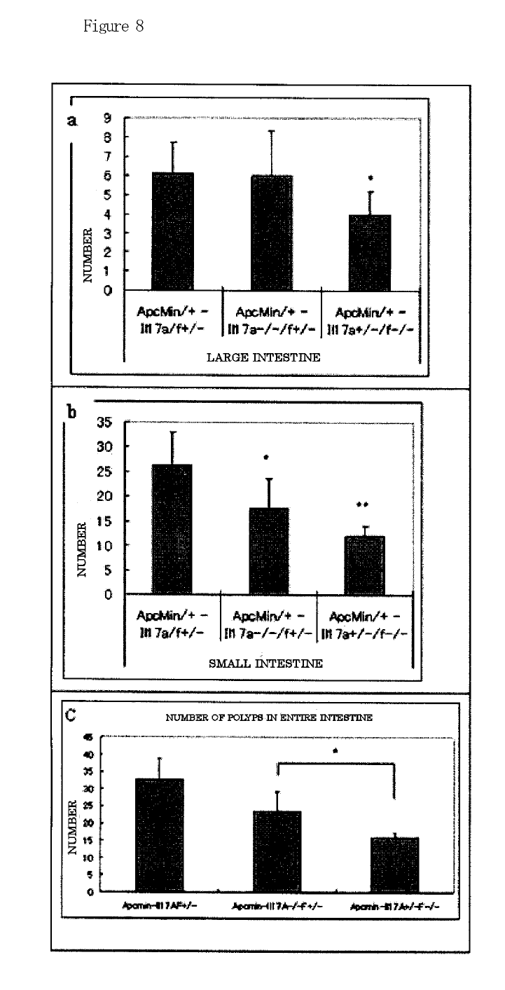

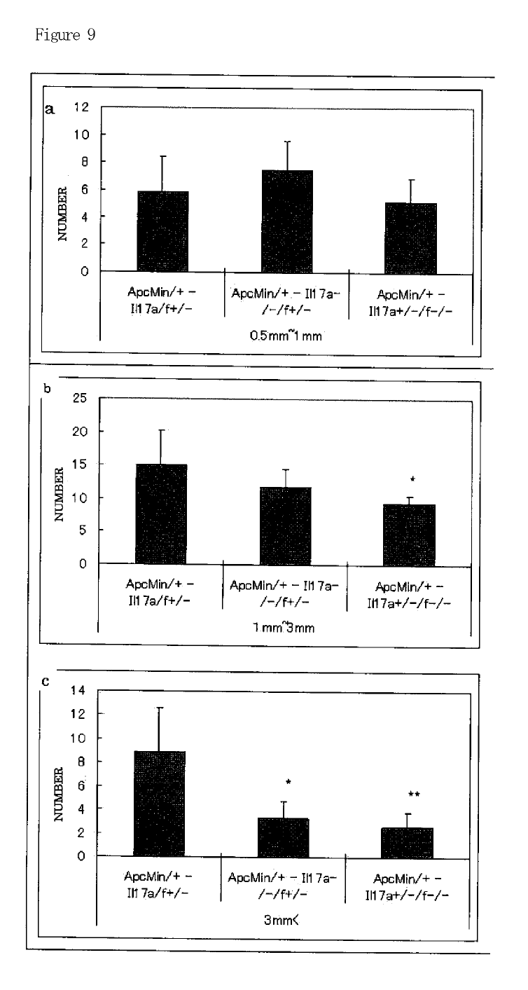

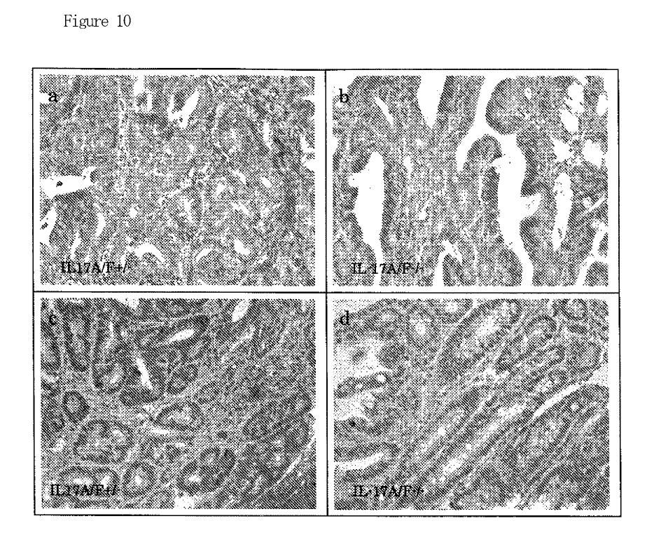

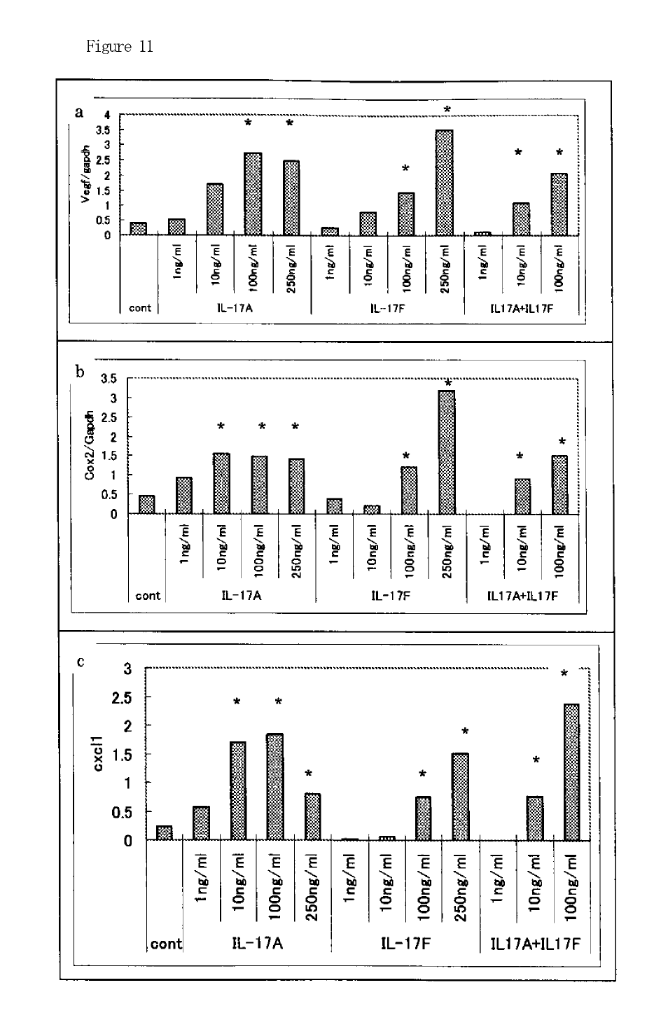

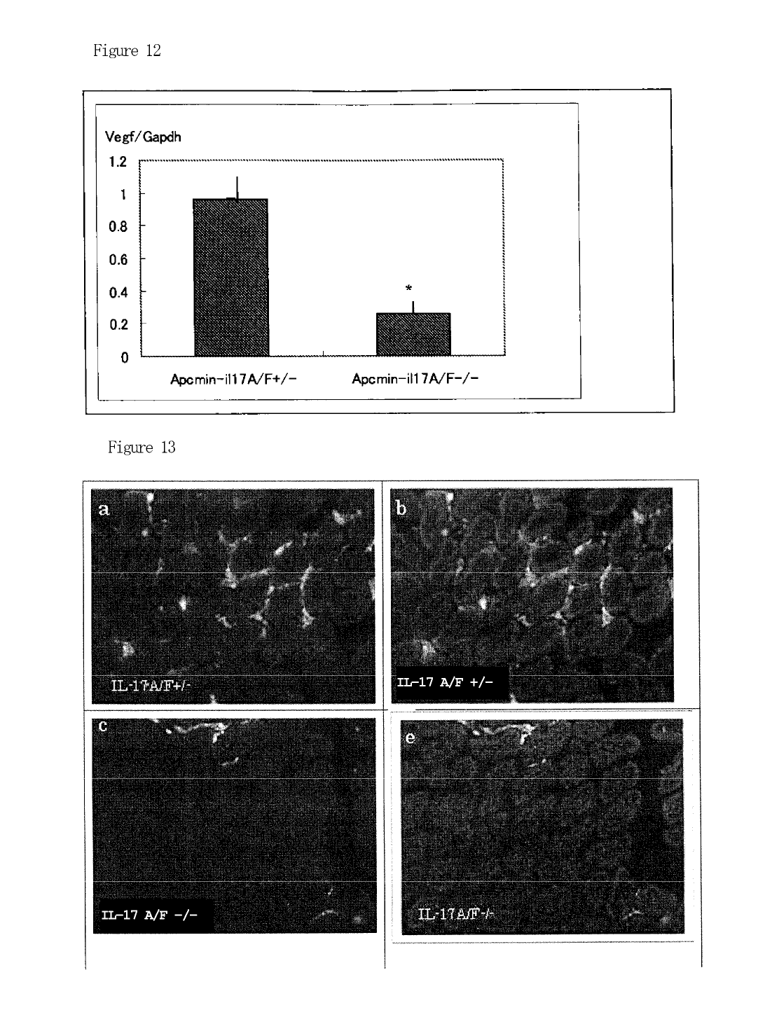

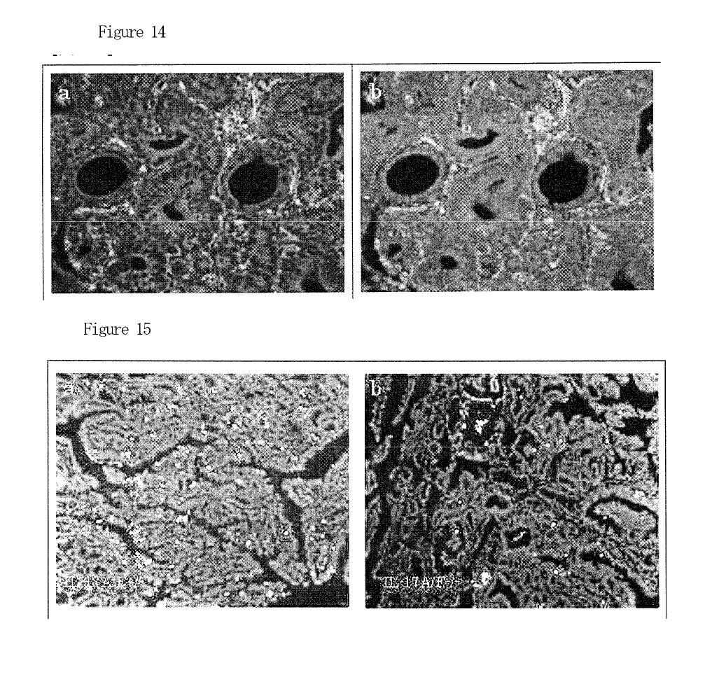

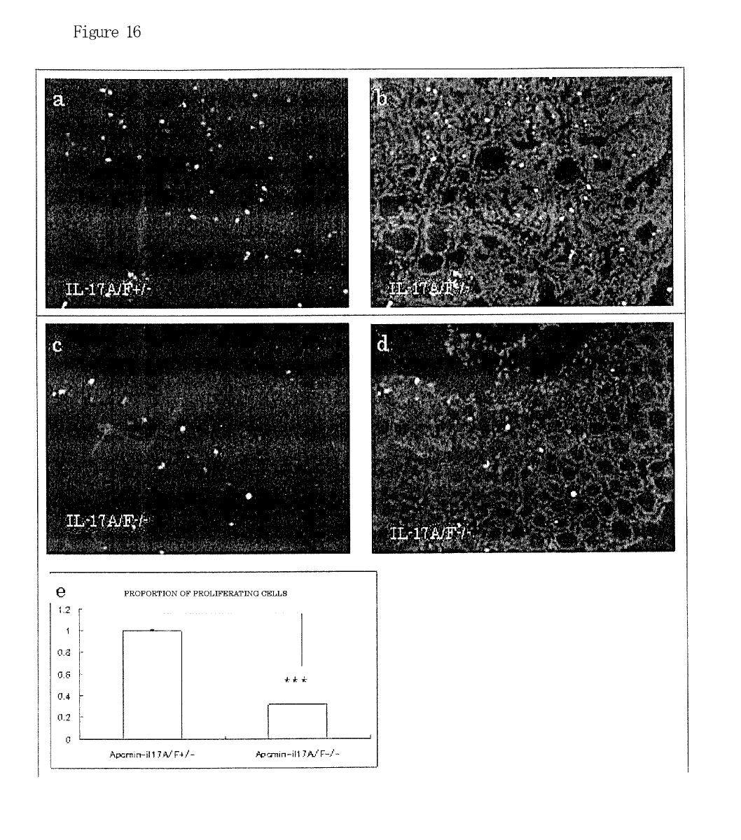

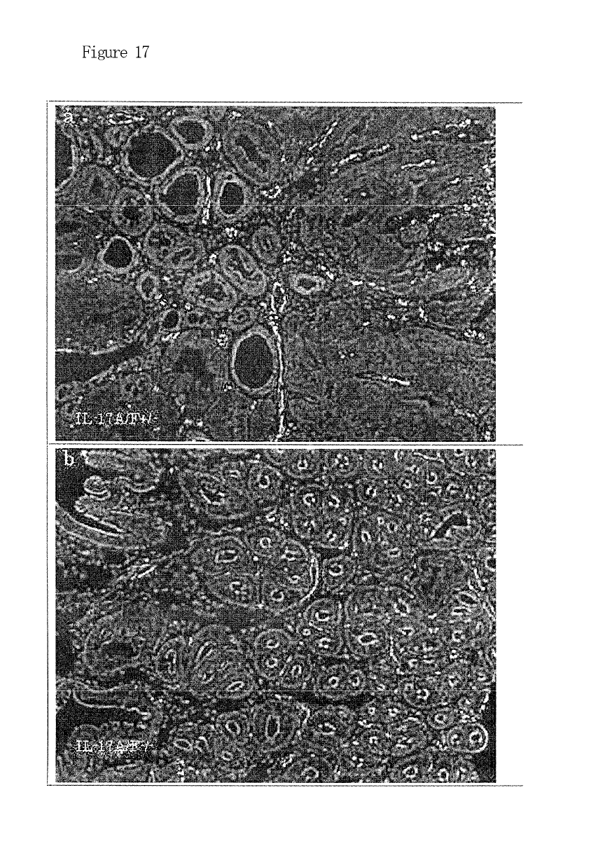

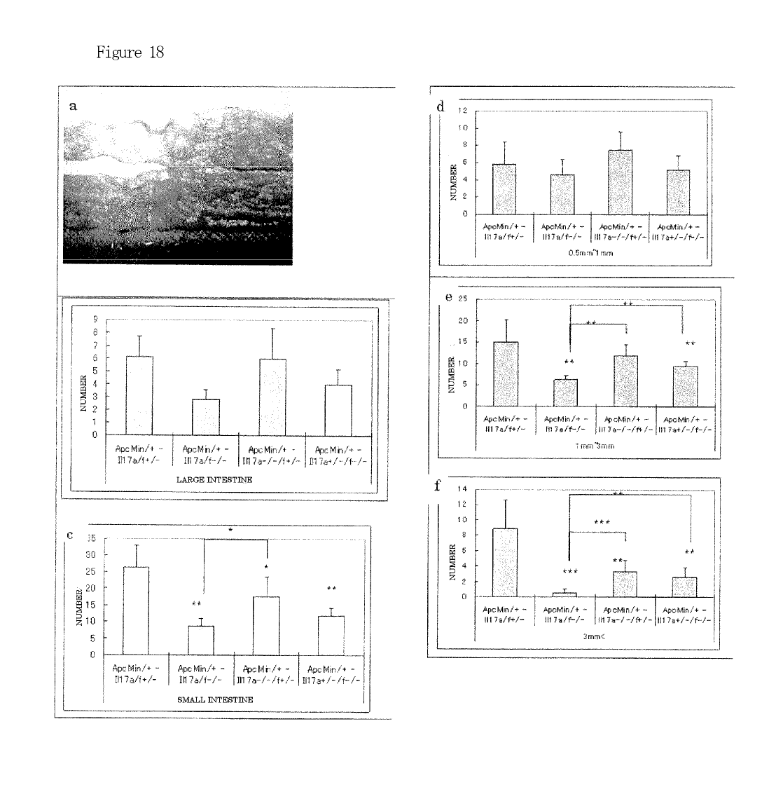

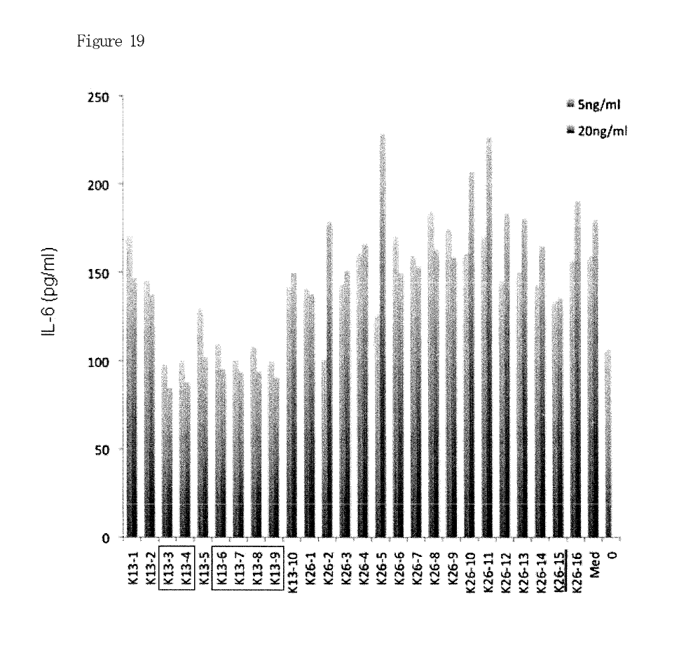

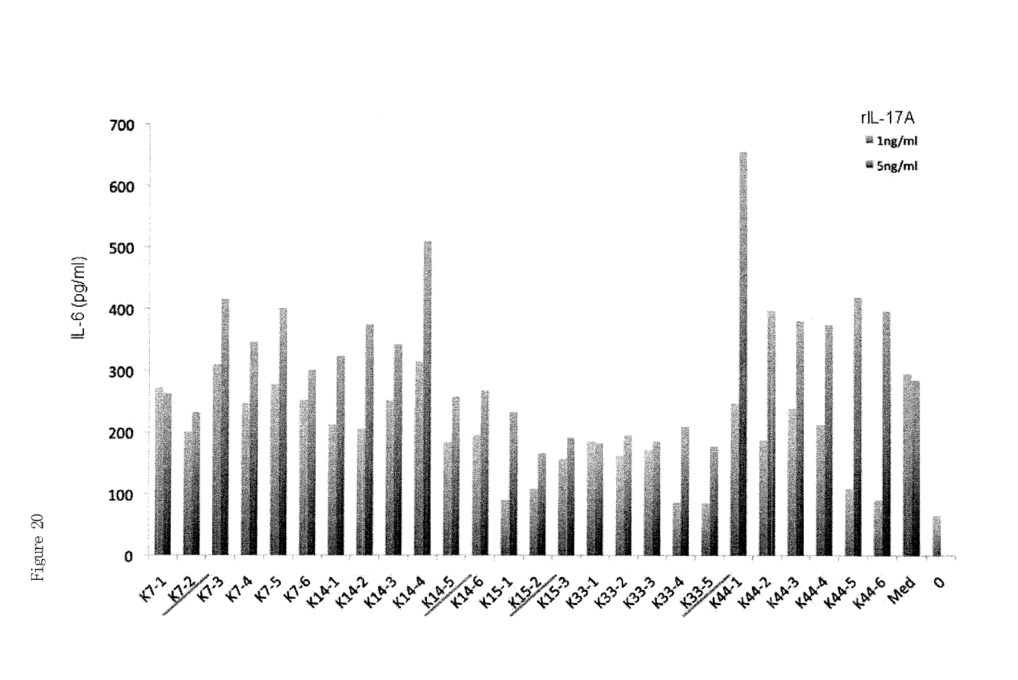

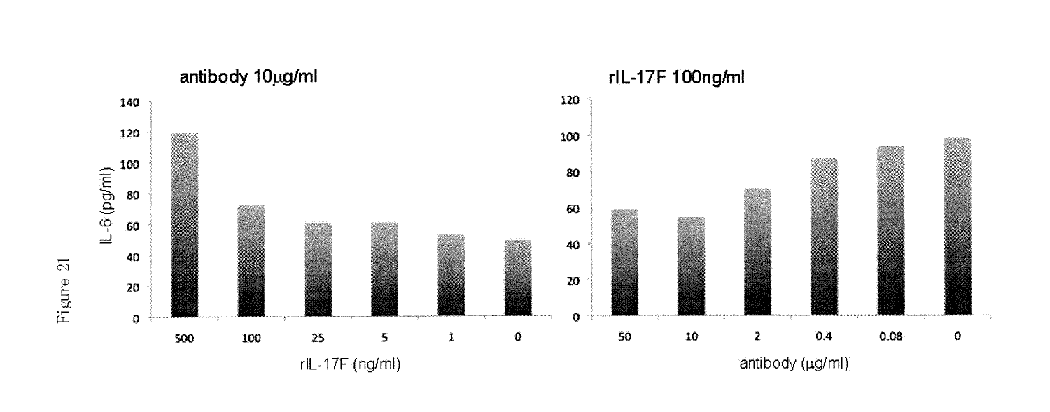

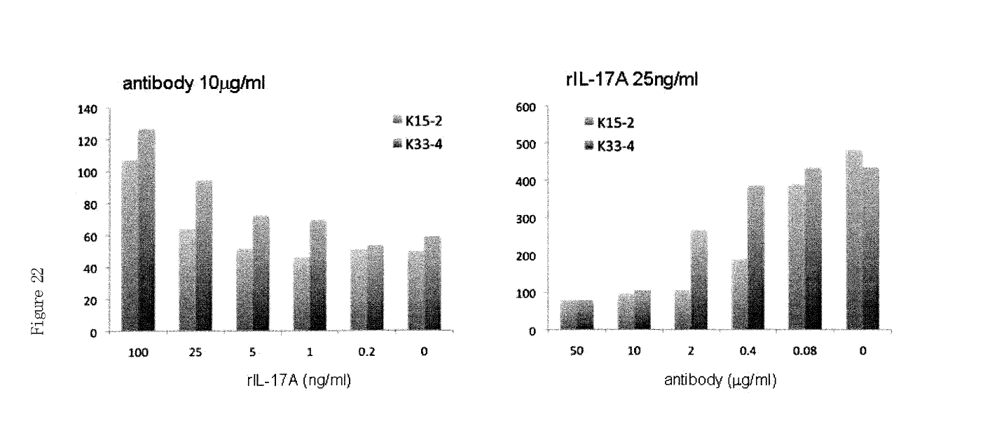

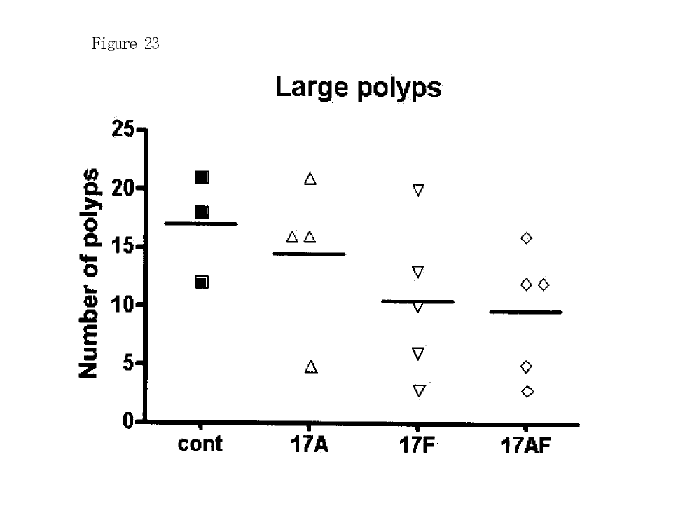

### BRIEF DESCRIPTION OF THE DRAWINGS

[FIG. 1] shows the state of the intestine in an Apc^{Min/+} mouse and an Apc^{Min/+}-Il1rn^{-/-} mouse (4.5 months old). The upper photograph is the Apc^{Min/+} mouse and the lower one is the Apc^{Min/+}-Il1rn^{-/-} mouse. The Apc^{Min/+}-Il1rn^{-/-} mouse was seen to have developed more polyps than the Apc^{Min/+} mouse. For the data, n=6 of both were examined, and the state of one typical sample was described.

[FIG. 2] shows a comparison of the number of polyps that developed in an Apc^{Min/+} mouse and an Apc^{Min/+}-Il1rn^{-/-} mouse. When compared divided into large intestine and small intestine regions, the Apc^{Min/+}-Il1rn^{-/-} mouse was seen to have developed more polyps in both regions (a). When the number of polyps that developed was investigated classified by size along the entire length of the intestine, no change could be found in the number of 0.5-1 mm polyps, but the number increased significantly above that level (b). For the data, n=3 Apc^{Min/+} mice and Apc^{Min/+}-Il1rn^{-/-} mice each were compared.

[FIG. 3] shows the results of microarray analysis in non-tumor parts and tumor parts of Apc^{Min/+} mice. When functional group analysis using GSEA was performed in non-tumor parts (WT_N) and tumor parts (WT_P) of Apc^{Min/+} mice, significant enhancement of the inflammatory pathway was demonstrated.

[FIG. 4] shows the results of microarray analysis in non-tumor parts and tumor parts of Apc^{Min/+}-Il1rn^{-/-} mice. When functional group analysis using GSEA was performed in non-tumor parts (RA_N) and tumor parts (RA_P) of Apc^{Min/+}-Il1rn^{-/-} mice (Non-patent Reference 39), significant enhancement of the pathway relating to the cell cycle of fibroblasts was demonstrated.

[FIG. 5] shows variations in the expression of Il17 family molecules by quantitative PCR. Although no difference in Il17a production could be seen in non-polyp parts and polyp locales in Apc^{Min/+} mice, expression was understood to be significantly increased in polyp locales in Apc^{Min/+}-Il1rn^{-/-} mice. A comparison of the polyp locales of the two also found significantly elevated expression in the Apc^{Min/+}-Il1rn^{-/-} mice (a). A difference in Il17f production was seen in the polyp locales in both Apc^{Min/+} mice and Apc^{Min/+}-Il1rn^{-/-} mice. A comparison of the polyp locales of the two also found significantly elevated expression in the Apc^{Min/+}-Il1rn^{-/-} mice (b).

[FIG. 6] shows variations in the expression of Cox2 by quantitative PCR. When expression of Cox2 was investigated in Apc^{Min/+} mice and Apc^{Min/+}-Il1rn^{-/-} mice using quantitative PCR, no significant difference could be found between the two. The study was conducted in n=3 each.

[FIG. 7] shows the state of the number of polyps that developed in Apc^{Min/+}-Il17a/f^{+/-} (Apc^{Min/+}-Il17a^{+/-}/f^{+/-}) mice and Apc^{Min/+}-Il17a^{-/-}/f^{+/-} mice and Apc^{Min/+}-Il17a^{+/-}/f^{-/-} mice. (a) shows a comparison of the state in Apc^{Min/+}-Il17a/f^{+/-} mice and Apc^{Min/+}-Il17a^{-/-}/f^{+/-} mice. (b) is a photograph showing a comparison of the state in Apc^{Min/+}-Il17a/f^{+/-} mice and Apc^{Min/+}-Il17a^{+/-}/f^{-/-} mice. Typical photographs are described from among a comparison of n=7 Apc^{Min/+}-Il17a/f^{+/-} mice, n=6 Apc^{Min/+}-Il17a^{-/-}/f^{+/-} mice, and n=5 Apc^{Min/+}-Il17a^{+/-}/f^{-/-} mice.

[FIG. 8] shows a comparison of the number of polyps that developed by site in Apc^{Min/+}-Il17a/f^{+/-} (Apc^{Min/+}-Il17a^{+/-}/f^{+/-}) mice and Apc^{Min/+}-Il17a^{-/-}/f^{+/-} mice and Apc^{Min/+}-Il17a^{+/-}/f^{-/-} mice. In the large intestine, while no significant difference could be found in the number of polyps that developed in Apc^{Min/+}-Il17a^{-/-}/f^{+/-} mice in comparison to Apc^{Min/+}-Il17a^{-/-}/f^{+/-} mice, the number of polyps that developed was significantly decreased in Apc^{Min/+}-Il17a^{+/-}/f^{-/-} mice (a). In the small intestine, the number of polyps that developed was decreased in both Apc^{Min/+}-Il17a^{-/-}/f^{+/-} mice and Apc^{Min/+}-Il17a^{+/-}/f^{-/-} mice in comparison to Apc^{Min/+}-Il17a/f^{+/-} mice (b). The number of polyps that developed throughout the entire intestine was significantly decreased in Apc^{Min/+}-Il17a^{+/-}/f^{-/-} mice in comparison to Apc^{Min/+}-Il17a^{-/-}/f^{+/-} mice (c). The comparison was conducted in n=7 Apc^{Min/+}-Il17a/f^{+/-} mice, n=6 Apc^{Min/+}-Il17a^{-/-}/f^{+/-} mice, and n=5 Apc^{Min/+}-Il17a^{+/-}/f^{-/-} mice.

[FIG. 9] shows a comparison of each size of polyp throughout the entire intestine in Apc^{Min/+}-Il17a/f^{+/-} (Apc^{Min/+}-Il17a^{+/-}/f^{+/-}) mice and Apc^{Min/+}-Il17a^{-/-}/f^{+/-} mice and Apc^{Min/+}-Il17a^{+/-}/f^{-/-} mice. In the results of a comparison of Apc^{Min/+}-Il17a/f^{+/-} mice and Apc^{Min/+}-Il17a^{-/-}/f^{+/-} mice, no significant difference could be seen in the number of polyps from 0.5 mm up to 3 mm that developed in Apc^{Min/+}-Il17a^{-/-}/f^{+/-} mice in comparison to Apc^{Min/+}-Il17a/f^{+/-} mice [(a) and (b)], but a significant decrease was confirmed in the number of polyps of 3 mm or larger (c). In contrast to this, when compared with Apc^{Min/+}-Il17a/f^{+/-} mice, no significant difference could be seen from 0.5 mm up to 1 mm in Apc^{Min/+}-Il17a^{+/-}/f^{-/-} mice (a), but a significant decrease was confirmed in the number of polyps of larger sizes that developed [(b) and (c)]. No significant difference could be seen when Apc^{Min/+}-Il17a^{-/-}/f^{+/-} mice and Apc^{Min/+}-Il17a^{+/-}/f^{-/-} mice were compared. The comparison was conducted in n=7 Apc^{Min/+}-Il17a/f^{+/-} mice, n=6 Apc^{Min/+}-Il17a^{-/-}/f^{+/-} mice, and n=5 Apc^{Min/+}-Il17a^{+/-}/f^{-/-} mice.

[FIG. 10] shows the difference in IL-17A- and IL-17F-producing cells in the polyp locale. The results of immunostaining IL-17A and IL-17F in Apc^{Min/+}-Il17a/f^{+/-} (Apc^{Min/+}-Il17a^{+/-}/f^{+/-}) mice and Apc^{Min/+}-Il17a/f^{-/-} (Apc^{Min/+}-Il17a^{-/-}/f^{-/-}) mice showed IL-17A to be produced by infiltrating cells (a) and IL-17F to be produced by epithelial cells as well as infiltrating cells (c). (b) and (d) show the results of immunostaining IL-17A and IL-17F, respectively, in Apc^{Min/+}-Il17a/f^{-/-} mice. For the data, testing was performed four separate times, and a typical results sheet was described.

[FIG. 11] shows variations in the expression of angiogenic factors by MEF in response to IL-17A and IL-17F stimulation using quantitative PCR. The expression of angiogenic factors (Vegfa, cox2, cxcl1) varied when mouse embryonic fibroblasts (MEF) were stimulated by IL-17A and IL-17F. Concentration-dependent elevation of expression of angiogenic factors was seen with both IL-17A and IL-17F. For the data, testing was performed three separate times, and the results were summarized.

[FIG. 12] shows a comparison of the amounts of Vegfa produced in the polyp locale in Apc^{Min/+}-Il17a/f^{+/-} (Apc^{Min/+}-Il17a^{+/-}/f^{+/-}) mice and Apc^{Min/+}-Il17a/f^{-/-} (Apc^{Min/+}-Il17a^{-/-}/f^{-/-}) mice using quantitative PCR. In the results obtained by comparing the amounts of Vegfa produced in the polyp locale in Apc^{Min/+}-Il17a/f^{+/-} mice and Apc^{Min/+}-Il17a/f^{-/-} mice, the amount produced was shown to be significantly decreased in Apc^{Min/+}-Il17a/f^{-/-} mice. Testing was conducted using n=4 of both mice.

[FIG. 13] shows immunostaining by VEGFA in the polyp locale in Apc^{Min/+}-Il17a/f^{+/-} (Apc^{Min/+}-Il17a^{+/-}/f^{+/-}) mice and Apc^{Min/+}-Il17a/f^{-/-} (Apc^{Min/+}-Il17a^{-/-}/f^{-/-}) mice. The drawing on the left is VEGFA stain; that on the right is nuclear stain. In the results of immunostaining in Apc^{Min/+}-Il17a/f^{+/-} mice and Apc^{Min/+}-Il17a/f^{-/-} mice, it was confirmed that VEGFA-producing cells are not epithelial cells. A comparison of the two also suggested that the amount of VEGFA produced decreases in Apc^{Min/+}-Il17a/f^{-/-} mice. For the data, testing was conducted using n=4 of both mice.

[FIG. 14] shows VIMENTIN immunostaining in Apc^{Min/+}-Il17a/f^{+/-} (Apc^{Min/+}-Il17a^{+/-}/f^{+/-}) mice. The drawing on the left is VIMENTIN stain; that on the right is nuclear stain. As a result of staining by VIMENTIN, which is a marker of fibroblasts, in Apc^{Min/+}-Il17a/f^{+/-} mice, it was understood that the majority of the stromal cells that construct the polyp are fibroblasts. For the data, testing was conducted in n=6, and a typical results sheet was described.

[FIG. 15] shows a comparison of apoptotic cells by TUNEL. Apoptotic cells were detected by TUNEL. The drawing on the left shows apoptotic cells; that on the right shows nuclei. Apc^{Min/+}-Il17a/f^{+/-} (Apc^{Min/+}-Il17a^{+/-}/f^{+/-}) mice are (a) and Apc^{Min/+}-Il17a/f^{-/-} (Apc^{Min/+}-Il17a^{-/-}/f^{-/-}) mice are (b). No significant difference could be seen between the two. Testing was conducted using n=6 of both mice.

[FIG. 16] shows a comparison of proliferating cells by immunostaining in Apc^{Min/+}-Il17a/f^{+/-} (Apc^{Min/+}-Il17a^{+/-}/f^{+/-}) mice and Apc^{Min/+}-Il17a/f^{-/-} (Apc^{Min/+}-Il17a^{-/-}/f^{-/-}) mice. The drawing on the left is proliferating cells among cells in the M phase of the cell cycle; that on the right shows the nuclei. The results in Apc^{Min/+}-Il17a/f^{+/-} mice are (a) and (b). The results in Apc^{Min/+}-Il17a/f^{-/-} mice are (c) and (d). A comparison of the two confirmed a significant decrease in proliferating cells in Apc^{Min/+}-Il17a/f^{-/-} mice (e). For the data, testing was conducted in n=6, and a typical sample was described.

[FIG. 17] shows the state of vascular cells in the polyp locale in Apc^{Min/+}-Il17a/f^{+/-} (Apc^{Min/+}-Il17a^{+/-}/f^{+/-}) mice and Apc^{Min/+}-Il17a/f^{-/-} (Apc^{Min/+}-Il17a^{-/-}/f^{-/-}) mice by immunostaining with anti CD31 antibody. A comparison of Apc^{Min/+}-Il17a/f^{+/-} mice and Apc^{Min/+}-Il17a/f^{-/-} mice showed a decrease in vascular volume in the Apc^{Min/+}-Il17a/f^{-/-} mice. For the data, testing was conducted in n=6 of both mice, and a typical type was described.

[FIG. 18] shows a comparison of Apc^{Min/+}-Il17a/f^{+/-} (Apc^{Min/+}-Il17a^{+/-}/f^{+/-}) mice and Apc^{Min/+}-Il17a/f^{-/-} (Apc^{Min/+}-Il17a^{-/-}/f^{-/-}) mice (6 months old). The state of polyp formation is shown in Apc^{Min/+}-Il17a/f^{+/-} mice and Apc^{Min/+}-Il17a/f^{-/-} mice (a). The photographs show the state of a typical sample as a result of confirmation in n=6. When polyp formation in Apc^{Min/+}-Il17a/f^{+/-} mice and Apc^{Min/+}-Il17a/f^{-/-} mice was compared by site, polyp formation in both the large intestine and small intestine was shown to be suppressed in Apc^{Min/+}-Il17a/f^{-/-} mice (b). When classified by size, no significant difference was seen in polyps 0.5-1 mm in size, but the formation of larger polyps was understood to be significantly suppressed in Apc^{Min/+}-Il17a/f^{-/-} mice (c). As a result of a comparison of Apc^{Min/+}-Il17a/f^{+/-} mice, Apc^{Min/+}-Il17a^{-/-}/f^{+/-} mice and Apc^{Min/+}-Il17a^{+/-}/f^{-/-} mice, polyps in the small intestine and the number of polyps 3 mm or larger in size were understood to be significantly decreased in the Apc^{Min/+}-Il17a/f^{-/-} mice in comparison to the respective single knockout mice. The data were confirmed in n=7 Apc^{Min/+}-Il17a/f^{+/-} mice, n=6 Apc^{Min/+}-Il17a/f^{-/-} mice, n=6 Apc^{Min/+}-Il17a^{-/-}/f^{+/-} mice, and n=5 Apc^{Min/+}-Il17a^{+/-}/f^{-/-} mice.

[FIG. 19] shows the results of evaluating the anti-IL-17F neutralizing activity after secondary screening. The IL-6 induction inhibiting activity by rIL-17F of each monoclonal antibody in MEF is shown.

[FIG. 20] shows the results of evaluating the anti-IL-17A neutralizing activity after secondary screening. The IL-6 induction inhibiting activity by rIL-17A of each monoclonal antibody in MEF is shown.

[FIG. 21] shows the IL-17F neutralizing activity of a purified anti-IL-17F antibody (clone K13-4). The IL-6 induction inhibiting activity by rIL-17F in MEF is shown.

[FIG. 22] shows the IL-17A neutralizing activity of purified anti-IL-17A antibodies (clones K15-2 and K33-4). The IL-6 induction inhibiting activity by rIL-17A in MEF is shown.

[FIG. 23] shows the number of large (3 mm or larger) polyps that developed in the large intestine after six once-a-week intraperitoneal administrations of mouse IgG (control), anti-mouse IL-17A antibody, anti-mouse IL-17F antibody, and both anti-mouse IL-17A antibody and anti-mouse IL-17F antibody to 4-month-old Apc^{Min/+} mice (C57BL/6J background).

### BEST MODE FOR CARRYING OUT THE INVENTION

As described above, the present invention provides an intestinal disease treatment that targets IL-1 family molecules and IL-17 family molecules, especially IL-17F, and a pharmaceutical for this treatment. No reports have suggested a relationship between IL-17F and cancer prior to the present invention. Therefore, the mechanism of action of IL-17F during carcinogenesis and the relationship between colorectal cancer and these cytokines at the time of spontaneous onset had not been studied.

Thus, the novel method of the present invention that treats intestinal disease by inhibiting IL family molecules, especially IL-17F, includes bringing a composition that contains a therapeutically effective quantity of an IL-17F inhibitor, typically an IL-17F antagonist capable of suppressing binding of an IL-17F receptor and IL-17F, or a substance capable of inhibiting the expression of IL-17F or an IL-17F receptor in tissues, into contact with a tissue that is developing or is at risk for developing intestinal polyps or cancer.

A. IL-17F Antagonist

An IL-17F antagonist is used in the present invention as a drug to inhibit the physiological effects of IL-17F in a tissue. These can take various forms, including compounds that interact with an IL-17F receptor or IL-17F so as to interfere with the natural functional interactions of IL-17F and IL-17F receptors. Examples of antagonists include monoclonal or polyclonal antibodies that produce an immune reaction with either IL-17F or an IL-17F receptor and mimetics of either IL-17F or an IL-17F receptor that mimic a structural region necessary in the ligand binding reaction of the IL-17F receptor.

Antibody:

One embodiment discloses an IL-17F antagonist that takes the form of a monoclonal antibody that reacts immunologically with IL-17F to suppress binding of natural IL-17F and an IL-17F receptor, as discussed in this specification. Methods of producing cell strains to produce such antibodies and methods of producing this monoclonal antibody can be carried out easily by those skilled in the art, and a preferred embodiment is also presented in the working examples.

Furthermore, the term “antibody” is used in this specification as a collective noun to indicate a population of immunoglobulin molecules and/or a population of immunologically active parts of immunoglobulins (in other words, molecules that contain an antibody binding site or paratope). The term “antibody binding site” means a structural part of an antibody molecule constructed from a variable or hypervariable region of a heavy chain and light chain that binds specifically with an antigen.

Typical antibodies used in the present invention are intact immunoglobulin molecules, essentially intact immunoglobulin molecules, and parts of immunoglobulin molecules containing a paratope (including fragments known as Fab, Fab′, F(ab′)₂, and F(v) in the art or parts termed antibody fragments). For example, antibody Fab and F(ab′)₂ parts (fragments) are prepared by proteolysis of an essentially intact antibody by papain and pepsin, respectively, in accordance with known methods (for example, refer to Theofilopolous & Dixon, U.S. Pat. No. 4,342,566). An Fab′ antibody fragment is also known and is produced from a F(ab′)₂ fragment by reducing the disulfide bond that joins two heavy chain fragments by mercaptoethanol, for example, and alkylating the protein mercaptan produced by a reagent such as iodoacetamide. Reference can be made to, for example, Morrison S L.: Two heads are better than one, Nat. Biotechnol., Vol. 25(11): 1233-4 (2007) with regard to other antibody-related inhibitors.

A “monoclonal antibody” consists of an antibody produced by a single cell clone called a hybridoma that typically secretes (produces) only one type of antibody molecule. This hybridoma cell is formed by fusing an antibody-producing cell and a myeloma or other self-perpetuating cell line. The preparation of such an antibody was first described by Kohler and Milstein (Kohler & Milstein, Nature 256: 495-497 (1975)). A separate method is also described by Zola (Zola, “Monoclonal Antibodies: A Manual of techniques)” CRC Press, Inc. (1987)).

However, since IL-17F is an endogenous molecule, antibody-producing cells can be acquired efficiently by using If17f^{-/-} mice, the production method for which is described in detail in “Ishigame et al., Immunity, Vol. 30, pp. 108-119 (2009)” (Non-patent Reference 26) and Supplemental Data (http://www.immunity.com/supplemental/51074-7613(08)00554-2), as immune animals when forming hybridomas that produce anti-mouse IL-17F antibody.

The supernatant of the hybridoma prepared in this way can be reacted immunologically with IL-17F and screened for the presence of a neutralizing antibody molecule that suppresses the binding of natural IL-17F to an IL-17F receptor. In sum, the neutralizing antibody screened in this way can be used as an IL-17F inhibitor of the present invention to suppress binding of natural IL-17F and an IL-17F receptor.

A method that employs IL-6 production by mouse embryonic fibroblasts (MEF) as an indicator can be given as a concrete example of the above neutralizing antibody screening. Specifically, MEF are known to produce IL-6 by IL-17F stimulation (Hu Y, Ota N, Peng I, Refino C J, Danilenko D M, Caplazi P, Ouyang W.: IL-17RC is required for IL-17A- and IL-17F-dependent signaling and the pathogenesis of experimental autoimmune encephalomyelitis., J. Immunol., Vol. 184(8): 4307-16 (2010)). This inhibition of IL-6 production can therefore be used to screen for anti-IL-17F antibody neutralizing activity. The details of this screening are described in the working examples.

Furthermore, a humanized monoclonal antibody provides particular advantages over a mouse monoclonal antibody especially when used therapeutically in humans. Specifically, a human antibody is not rapidly eliminated from the blood circulation as is a foreign antigen and the immune system is not activated in the same form as by a foreign antigen and foreign antibody. Methods of preparing humanized antibodies are generally known in the art and can be applied easily to the antibody of the present invention.

Mimetic:

A typical “mimetic” of the present invention has an amino acid sequence characteristic of either IL-17F itself or an IL-17F receptor in a region necessary to the interaction of IL-17F and a receptor thereof and may be a polypeptide that exhibits IL-17F antagonist activity. An IL-17F mimetic can be designed using any of the various structural analysis methods already known in the art for drug design. These analysis methods include molecular modeling, two-dimensional nuclear magnetic resonance (2-D NMR) analysis, x-ray crystallography, random screening of peptides, peptide analog or other chemical polymer libraries, and similar drug design methods.

Preferred IL-17F antagonists having selectivity for IL-17F can be distinguished easily, for example, by IL-6 production inhibition assay by the above-mentioned MEF. For example, it will be appreciated that a mimetic can be used for the purposes of the present invention as long as it is a peptide containing the necessary amino acid sequence and can function, for example, as an IL-17F antagonist by assay as discussed in this specification. A mimetic polypeptide can take the form of any of various peptide derivatives; these include amides, conjugates with proteins, polymer peptides, fragments, chemically modified peptides, and similar derivatives. The term “chemically modified” means a polypeptide having one or more residues chemically derived by reaction of a functional side-chain group. Such derivative molecules include, for example, molecules in which a free amino group has been induced to form a carbobenzoxy group, t-butyloxycarbonyl group, chloroacetyl group, or formyl group. A free carboxy group can be induced to form a salt, methyl and ethyl ester, or other type of ester. A free hydroxy group can be induced to form an o-acyl or o-alkyl derivative. Also included as chemical derivatives are peptides containing one or more amino acid derivatives of the 20 naturally occurring types of standard amino acids.

B. IL-17F or IL-17F Receptor Expression Inhibitor

The LF-17F inhibitors of the present invention include substances capable of inhibiting the expression of IL-17F or an IL-17F receptor in a tissue. A siRNA molecule or antisense RNA molecule having IL-17F (or a receptor thereof) as its target can serve as a typical expression inhibitor.

siRNA Molecule:

siRNA (short interfering RNA) of the present invention is preferably double-stranded RNA that joins RNA (antisense RNA chain) complementary to a target sequence that is a transcription product (mRNA) of an IL-17F gene and RNA (sense RNA chain) complementary to this RNA. The sequences of transcription products of the IL-17F gene of the present invention are well known to those skilled in the art. siRNA for mouse IL-17F is also available from Santa Cruz Biotechnology, Inc. as “IL-17F siRNA(m): sc-146204.”

Generally, when siRNA is introduced into a cell, an RNAi phenomenon occurs, and RNA having a homologous sequence is degraded. siRNA of the present invention includes, in addition to siRNA itself (double-stranded RNA), shRNA (short hairpin RNA), dsRNA (double strand RNA), and expression vectors capable of expressing these so as to produce this siRNA and may be of any form as long as it is capable of triggering RNAi. This siRNA is one that has been chemically synthesized artificially, one that has been modified, one that has been biochemically synthesized, one that has been synthesized within a living organism, or one produced by degradation of double-stranded RNA of approximately 40 or more bases in an organism and is double-stranded RNA of 10 or more base pairs. The number of bases in the siRNA is generally 10-30, preferably 15-25, and more preferably 19-23. This siRNA also usually has a 5′-phosphoric acid, 3′-OH structure, and approximately two bases preferably project at the 3′ end (Elbashir S M, Harborth J, Lendeckel W, Yalcin A, Weber K, Tuschl T. Duplexes of 21-nucleotide RNAs mediate RNA interference in cultured mammalian cells. Nature. 2001 May 24; 411(6836): 494-8). The siRNA becomes single-stranded, and the other strand (guide strand) forms an RISC(RNA-induced-silencing-complex). The RISC recognizes and binds to mRNA having a sequence complementary to the guide strand and cleaves the mRNA at the center of the siRNA. In this way, siRNA can suppress its expression by degrading the mRNA of the gene that serves as its target.

Antisense RNA:

“Antisense” nucleic acids include nucleotide sequences complementary to the “sense” nucleic acids that encode a protein, for example, complementary to a double-stranded cDNA coding chain or complementary to an mRNA sequence. Therefore, the antisense nucleic acids can hydrogen bond to the sense nucleic acids. The antisense nucleic acids can be complementary to the entire IL-17F coding chain or to only a fragment thereof. The antisense oligonucleotides can be, for example, approximately 5, 10, 15, 20, 25, 30, 35, 40, 45, or 50 nucleotides long. The antisense nucleic acids of the present invention can be constructed by methods known in the art using chemical synthesis and enzyme ligation reactions. As another method, antisense nucleic acids can also be manufactured biologically using an expression vector in which the nucleic acids are subcloned in the antisense position.

The antisense RNA molecule of the present invention typically hybridizes or binds with the intracellular mRNA and/or genomic DNA that codes IL-17F, thereby inhibiting expression of the polypeptide by inhibiting transcription and/or translation.

C. IL-17A Inhibitor

As was mentioned above, no reports prior to the invention have suggested a relationship between IL-17F and cancer. Many studies to date have also indicated that the effect of IL-17F is weaker than that of IL-17A. However, the present inventors were surprised to obtain findings that suggested that this IL-17F plays a central role in tumorigenesis through the excessive production of IL-17F in the tumor locale in the actual pathogenesis of colorectal cancer. On the other hand, the present inventors confirmed that IL-17A also acts on fibroblasts and promotes angiogenesis. Inhibition of this IL-17A was also proven to decrease the number of colonic polyps that develop, and this effect was proven to be further potentiated by the joint use of IL-17F inhibition. Therefore, the novel method of the present invention to treat intestinal disease by inhibiting IL family molecules encompasses the combined use of an IL-17F inhibitor and an IL-17A inhibitor. Furthermore, the phrase “combined use” intends the simultaneous or sequential (in other words, each at separate times) administration of an IL-17F inhibitor and an IL-17A inhibitor by the same or different routes of administration. Therefore, the form of the two drugs is also not particularly restricted; the two may contained in the same unit dosage or in separate unit dosages.

Everything stated above regarding the IL-17F inhibitor can also be applied to the IL-17A inhibitor of the present invention. Specifically, the IL-17A inhibitor is also typically an IL-17A antagonist capable of suppressing the binding of an IL-17A receptor and IL-17A or a substance capable of inhibiting the expression of IL-17A or an IL-17A receptor in a tissue. The explanations given for IL-17F are also true for IL-17A antagonists and expression inhibitors.

Preferred examples of IL-17A inhibitors include anti-IL-17A monoclonal antibodies. An anti-human IL-17 (IL-17A) antibody to antagonize IL-17A is known from the International Publication WO2007/117749 pamphlet (Patent Reference 1), but methods of preparing cell lines to produce such antibodies and methods of producing these monoclonal antibodies can be conducted easily by persons skilled in the art, and preferred embodiments also appear in the working examples.

In sum, this monoclonal antibody may be prepared based on the method described by Kohler and Milstein (Kohler & Milstein, Nature 256: 495-497 (1975)) or the method described by Zola (Zola, “Monoclonal Antibodies: A Manual of techniques” CRC Press, Inc. (1987)).

However, since IL-17A is also an endogenous molecule, antibody-producing cells can also be acquired efficiently by using I17a^{-/-} mice, the production method of which is described in detail in “Nakae et al., Immunity, Vol. 17, pp. 375-387 (2002)” (Non-patent Reference 38), as immune animals when forming hybridomas that produce anti-mouse IL-17A antibody.

The supernatant of the hybridoma prepared in this way can be reacted immunologically with IL-17A and screened for the presence of a neutralizing antibody molecule that suppresses the binding of natural IL-17A to an IL-17A receptor. In sum, the neutralizing antibody screened in this way can be used as an IL-17A inhibitor of the present invention to suppress binding of natural IL-17A and an IL-17A receptor.

A method that employs IL-6 production by mouse embryonic fibroblasts (MEF) as an indicator can be given as a concrete example of the above neutralizing antibody screening. Specifically, MEF are known to produce IL-6 by IL-17F stimulation (Hu Y, Ota N, Peng I, Refino C J, Danilenko D M, Caplazi P, Ouyang W.: IL-17RC is required for IL-17A- and IL-17F-dependent signaling and the pathogenesis of experimental autoimmune encephalomyelitis., J. Immunol., Vol. 184(8): 4307-16 (2010)). This inhibition of IL-6 production can therefore be used to screen for anti-IL-17F antibody neutralizing activity. The details of this screening are described in the working examples.

D. Method for Treating Intestinal Disease and Pharmaceutical Composition for Treatment of Intestinal Disease

As has been clarified above, the novel method of the present invention to treat intestinal disease includes bringing a pharmaceutical composition containing a therapeutically effective amount of an IL-17F inhibitor into contact with a tissue that is developing or is at risk for developing intestinal disease. Intestinal diseases that are the object of treatment of the present invention typically include intestinal tumors; and these tumors include polyps and cancer. The tumors treated by the method and pharmaceutical composition of the present invention can also typically be present in the colon. These colonic tumors include malignant epithelial tumors, carcinoid tumors, non-epithelial tumors, lymphoma, metastatic tumors, benign epithelial tumors, and neoplastic lesions (such as hyperplastic polyps and the like).

In producing the pharmaceutical composition of the present invention, it is preferable to make a pharmaceutical composition by adding pharmacologically acceptable auxiliary components as needed to the IL-17F inhibitor that is the active ingredient (furthermore, all explanations here also apply to IL-17A inhibitors). However, it is preferable to adapt the selection of auxiliary components and mixture with the active ingredient so that interactions do not substantially lower the pharmaceutical efficacy of the IL-17F inhibitor under conditions of ordinary use. As shall be apparent, the pharmacologically acceptable auxiliary components are also preferably of high enough purity and low enough toxicity that they do not pose any problems in terms of safety when administered to humans. Examples of pharmacologically acceptable auxiliary components include sugars, starch, cellulose derivatives, gelatin, stearic acid, magnesium stearate, vegetable oil, polyols, alginic acid, isotonizing agents, buffers, wetting agents, lubricants, coloring agents, flavorings, preservatives, stabilizers, antioxidants, antiseptics, antimicrobials, and the like.

Examples of the drug form of the pharmaceutical composition of the present invention include an injection, rectally-absorbed agent, orally-administered agent, and the like. However, the specific dosage forms of these are in no way limited.

For example, when the pharmaceutical composition of the present invention is administered as an injection, it is preferably adapted to intramuscular, subcutaneous, or intravenous administration. When administered as a rectally-absorbed agent, it generally takes the form of a suppository. When administered as an orally-administered agent, it can take a form for oral use such as a liposome formulation, microcapsule formulation, and the like.

As a more concrete example, when the pharmaceutical composition of the present invention is formulated as an injection, the desired injection can be prepared, for example, by dissolving an anti-IL-17F antibody in distilled water for injection in which have been dissolved suitable amounts of buffer, isotonizing agent, and pH adjuster, sterilizing by passage through a sterilizing filter, and dispensing into ampules.

When the pharmaceutical composition of the present invention is formulated as a rectally absorbed agent, a suppository can be obtained, for example, by appropriate selection and use of an anti-IL-17F antibody, an absorption accelerator having chelating capacity, such as sodium pectate, sodium alginate, or the like, and a hypertonizing agent, such as sodium chloride, glucose, or the like, and dissolution or dispersion of these in distilled water or an oily solvent (refer to UKP 2092002 and 2095994).

When the pharmaceutical composition of the present invention is formulated as an orally administered agent, an anti-IL-17F antibody can be made into a tablet, fine granule, granule, suspension, or capsule together with a known, pharmacologically acceptable excipient, binder, lubricant, fluidity promoter, coloring agent, and other such carriers.

The therapeutically effective dose of IL-17F inhibitor, for example, anti-IL-17F antibody, contained as the active ingredient in the pharmaceutical composition of the present invention varies depending on the age, physique, gender, healthfulness of the subject, relative activity of the IL-17F inhibitor administered, drug form, administration frequency, and the like, but is, for example, from approximately 0.05 mg to approximately 20 mg per kilogram of body weight, more usually from approximately 0.1 mg to approximately 5 mg per kilogram of body weight. The frequency of administration also depends on the age, physique, gender, healthfulness of the subject, relative activity of the IL-17F inhibitor administered, dose, drug form, and the like, but may be in a range of from once/week to three times/day, preferably from once/week to once/day, and more preferably once/week or once/day.

Since the active ingredient of the pharmaceutical composition of the present invention does not interact with other drugs, it can be used in combination with various drugs to match the situation of the subject. Examples of drugs that can be used in combination include those listed in the International Publication WO2007/117749 pamphlet (Patent Reference 1).

The present invention is explained based on working examples. However, the scope of the present invention is not limited to the following examples.

### WORKING EXAMPLES

### Example 1

### Role of IL Family Molecules in the Pathogenesis of Colorectal Cancer

<Materials and Method>

1) Mice

For Apc^{Min/+} mice, mice with a C57BL/6J background were purchased from Jackson Laboratories.

Il1rn^{-/-} mice were produced by the method of “Horai et al., J. Exp. Med., Vol. 187, pp. 1463-1475 (1998)” (Non-patent Reference 37). Individuals back crossed at least eight generations with C57BL/6J (Japan SLC) were used in the following studies.

Il17a^{-/-} mice were produced according to “Nakae et al., Immunity, Vol. 17, pp. 375-387 (2002)” (Non-patent Reference 38) by substituting exon 1-2 containing an ATG initiation codon by a neomycin resistance gene on an ES cell. Individuals back crossed at least eight generations with C57BL/6J (Japan SLC) were used in the following studies.

Il17f^{-/-} mice were produced according to “Ishigame et al., Immunity, Vol. 30, pp. 108-119 (2009)” (Non-patent Reference 26) by substituting a hybromycinmycin resistance gene for exon 2-3 using Il17^{+/-} ES cells. Individuals back crossed at least eight generations with C57BL/6J (Japan SLC) were used in the following studies.

Apc^{Min/+}-Il1rn^{-/-} mice, Apc^{Min/+}-Il17a^{-/-} mice, Apc^{Min/+}Il17f^{-/-} mice, Apc^{Min/+}-Il17a^{-/-}/f^{-/-} mice, Apc^{Min/+}-Il17a^{-/-}/f^{+/-} mice, Apc^{Min/+}-Il17a^{+/-}/f^{-/-} mice, and Apc^{Min/+}Il17a^{+/-}/f^{+/-} mice were produced by crossing the above mice. Furthermore, the mice were kept in an SPF environment in the Center for Experimental Medicine, The Institute of Medical Science, The University of Tokyo. All of the studies were conducted in accordance with the Institute of Medical Science animal experimentation manual and laws concerning the use of genetically-modified organisms, and the like.

2) Comparison of Polyp Formation

The intestines were removed from Apc^{Min/+} mice and Apc^{Min/+}-Il1rn^{-/-} mice at the age of 4.5 months and fixed by 10% neutral buffered formalin. Sizes of from 0.5 mm to 1 mm, from 1 mm to 3 mm, and larger than 3 mm were classified under the microscope, and the number of polyps that developed in the large intestine and small intestine was measured.

The intestines were removed from Apc^{Min/+}-Il17a^{-/-}/f^{-/-} mice, Apc^{Min/+}-Il17a^{-/-}/f^{+/-} mice, Apc^{Min/+}-Il17a^{+/-}/f^{-/-} mice, and Apc^{Min/+}Il17a^{+/-}/f^{+/-} mice at the age of six months and fixed by 10% neutral buffered formalin. Sizes of from 0.5 mm to 1 mm, from 1 mm to 3 mm, and larger than 3 mm were classified under the microscope, and the number of polyps that developed in the large intestine and small intestine was measured.

3) Extraction of mRNA

Polyp parts and non-polyp parts were collected from each mouse, and the mRNA was isolated by isopropanol precipitation after extraction by Sepasol RNA I Super (Nacalai Tesque Co., Ltd.). Furthermore, the study was conducted by making the polyp size uniform at from 2 mm to 3 mm. The cell line was also subjected to the same Sepasol RNA I Super (Nacalai Tesque Co., Ltd.) protocol. Furthermore, the cell line (MEF) was prepared to 1×10⁶, then cultured for three hours in RPMI medium containing antibiotics (penicillin and streptomycin). The cells were recovered three hours after adding IL-17A (R&D Co., Ltd.) or IL-17F (R&D Co., Ltd.) to make 1 ng/mL, 50 ng/mL, 100 ng/mL, or 250 ng/mL each.

4) Cell Line

MEF (mouse embryonic fibroblasts) were produced from embryonic (14.5 days) C57CL/6J mice. Cells cultured in DMEM (GIBCO Co.) with 10% FCS and antibiotics (penicillin and streptomycin) added were used as first-generation cultured cells.

5) DNA Microarray Analysis

Microarray analysis was performed using a Mouse Genome 430 2.0 Array (AFFYMETRIX Inc.) chip on a total of four types of mRNA from polyp and non-polyp parts of Apc^{Min/+} mice and polyp and non-polyp parts of Apc^{Min/+}-Il1rn^{-/-} mice. Functional group analysis was also performed using the analysis software “GSEA” on the polyp and non-polyp parts of Apc^{Min/+} mice and polyp and non-polyp parts of Apc^{Min/+}-Il1rn^{-/-} mice (Non-patent Reference 39).

6) Analysis by Quantitative PCR

The mRNA extracted in 3) above was adjusted to 50 ng/μL, then transcribed into cDNA using a High Capacity cDNA RT kit (made by Applied Biosystems Inc.). Quantitative PCR was then performed using a SYBRE kit (Takara Bio Inc.). The expression level was corrected using Gapdh, a housekeeping gene.

7) Preparation of Tissue Sections

The polyps sampled in 2) above were fixed for one hour by 10% neutral buffered formalin, then embedded in paraffin using an automatic embedding machine. Tissue sections were subsequently prepared by slicing to 5 μm.

8) Detection of Apoptotic Cells by TUNEL

The paraffin was removed from the tissue sections prepared in 7) above using xylene and ethanol, and apoptotic cells were then detected by TUNEL using an apoptosis detection kit (Roche Inc.).

9) Staining by Immunostain

Tissue sections prepared in 7) above were immunostained. After removing the paraffin using xylene and ethanol, the antigen was activated by 0.1M citrate buffer (pH 6). Blocking was performed for one hour by 2% goat serum (VECOTR)/PBS, and the primary antibody was reacted overnight using Vegf alfa (abcam Inc. Vimentin (abcam Inc.), phosphotilation Histn H (PH) 3 (abcam Inc.), CD31 (abcam Inc.), IL-17A (Santa Cnuz Biotechnology Inc.), and IL-17F (R&D Co., Ltd.). The secondary antibody was reacted for one hour using Alexa (Molecular Probe Inc.), Cy3 (Jackson Inc.), and streptavidin (Perkin Elmer Inc.). Hoechest (Molecular Probe Inc.) and DAB (Nacalai Tesque Co., Ltd.) were used as nuclear stains. Furthermore, a Biorevo (Keyence Corp.) was used in all examinations, and BZ-II (Keyence Corp.) was used in analysis. Furthermore, IL-17A and IL-17F were immunostained using a TSA system (Perkin Elmer Inc.), which is a tyramide amplification method.

10) Statistical Evaluation

All of the results obtained were evaluated statistically by Student's t-test. Furthermore, significant differences were designated as *: p<0.05, **: p<0.01, ***: p<0.001.

<Results>

1. Comparison of Apc^{Min/+} Mice and Apc^{Min/+}-Il1rn^{-/-} Mice (4.5 Months Old)

When the number of polyps in Apc^{Min/+} mice and Apc^{Min/+}-Il1rn^{-/-} mice was measured, the number of polyps increased predominantly in Apc^{Min/+}-Il1rn^{-/-} mice in comparison to Apc^{Min/+} mice (FIGS. 1 and 2). Enhancement of the inflammatory signaling pathway was seen in the polyp parts of Apc^{Min/+} mice in the results of microarray analysis (FIG. 3). The non-tumor parts and tumor parts of Apc^{Min/+}-Il1rn^{-/-} mice also showed an increase in factors related to the cell cycle of fibroblasts as a result of comparison of the microarray analysis data with the data in the literature (FIG. 4). However, no significant difference could be seen in Cox2 expression as a result of a comparison of the polyp parts of Apc^{Min/+} mice and Apc^{Min/+}-Il1rn^{-/-} mice in the results of analysis by quantitative PCR (FIG. 6). Similarly, Il17f production was significantly enhanced in the polyp parts of Apc^{Min/+} mice, and Il17f production was understood to be significantly enhanced when the polyp parts of Apc^{Min/+}-Il1rn^{-/-} mice and Apc^{Min/+} mice were compared in the results of analysis by quantitative PCR (FIG. 5a). No changes could be seen in the amount of Il17a produced in either the polyp parts or non-polyp parts in Apc^{Min/+} mice, but Il17a production was understood to be significantly enhanced when the polyp parts of Apc^{Min/+}-Il1rn^{-/-} mice and Apc^{Min/+} mice were compared (FIG. 5b).

The above results suggested that a state of excessive inflammation due to IL-1 family molecules exacerbates colorectal cancer by a pathway independent of COX2. A comparison of changes in gene expression by microarray of Apc^{Min/+}-Il1rn^{-/-} mice also showed a relationship between changes in the expression of pathways relating to the cell cycle of fibroblasts and IL-1 family molecules.

2. Comparison of Polyp Formation in Apc^{Min/+}-Il17a^{-/-}/f^{+/-} Mice, Apc^{Min/+}-Il17a^{+/-}/f^{-/-} Mice, and Apc^{Min/+}Il17a^{+/-}/f^{+/-} Mice (6 Months Old)

In the results of comparison of the number and size of polyps in Apc^{Min/+}-Il17a^{-/-}/f^{+/-} mice, Apc^{Min/+}-Il17a^{+/-}/f^{-/-} mice, and Apc^{Min/+}/Il17a^{+/-}/f^{+/-} mice, the decrease in polyps was less in Apc^{Min/+}-Il17a^{-/-}/f^{+/-} mice and Apc^{Min/+}-Il17a^{+/-}/f^{-/-} mice than in Apc^{Min/+}-Il17a^{-/-}/f^{-/-} mice, but the number of polyps 3 mm or larger was understood to be significantly decreased in comparison to Apc^{Min/+}-Il17a^{-/-}/f^{+/-} mice (FIGS. 7, 8, and 9). The number of 1 mm to 3 mm polyps was also shown to be decreased in Apc^{Min/+}-Il17a^{+/-}/f^{-/-} mice (FIG. 9). A significant decrease in polyp number was seen only in Apc^{Min/+}-Il17a^{+/-}/f^{-/-} mice in the results of comparison of the total number of polyps that developed in the intestines of Apc^{Min/+}-Il17a^{-/-}/f^{+/-} mice and Apc^{Min/+}-Il17a^{+/-}/f^{-/-} mice (FIG. 8c).

3. Identification of IL-17- and IL-17F-Producing Cells

As a result of immunostaining to identify IL17- and IL-17F-producing cells in tissue sections of Apc^{Min/+}-Il17a^{+/-}/f^{+/-} mice and Apc^{Min/+}-Il17a^{-/-}/f^{-/-} mice, it was understood that while cells that produce IL-17 are mainly infiltrating cells, cells that produce IL-17F are epithelial cells and cancer cells themselves in addition to infiltrating cells (FIG. 10).

4. Measurement of Angiogenesis Factors by Mouse Embryonic Fibroblasts (MEF)

Angiogenesis factors were understood to increase dependent on the concentrations of IL-17 and IL-17F as a result of adding IL-17 and IL-17F to MEF and measuring the expression of the angiogenesis factors Vegfa, Cxcl1, and Cox2 using quantitative PCR (FIGS. 11a, b, and c).

These results show that IL-17 family molecules enhance angiogenesis and promote tumorigenesis. The predicted mechanism is that IL-17A and IL-17F act on fibroblasts in the tumor milieu, thereby creating blood vessels in the tumor locale by accelerating the expression of factors that participate in angiogenesis such as VEGFA, CXCL1, and COX2 and producing an environment favorable to cancer cell growth.

5. Comparison of Polyp Environment in Apc^{Min/+}Il17a^{+/-}/f^{+/-} Mice and Apc^{Min/+}-Il17a^{-/-}/f^{-/-} Mice

When expression of Vegfa was studied using quantitative PCR, the level of Vegfa expression was understood to be decreased in the polyp parts of Apc^{Min/+}-Il17a^{-/-}/f^{-/-} mice in comparison to the polyp parts of Apc^{Min/+}-Il17a^{+/-}/f^{+/-} mice (FIG. 12). The results of staining VEGFA by immunostaining confirmed VEGF to be produced by infiltrating cells rather than epithelial cells (FIG. 13). Staining by Vimentin, a fibroblast marker, was therefore conducted to investigate the types of infiltrating cells in the polyp locale. As a result, the majority of the infiltrating cells were understood to be fibroblasts (FIG. 14).

6. Proliferation and Apoptotic Response in the Polyp Environment of Apc^{Min/+}-Il17a^{+/-}/f^{+/-}, Apc^{Min/+}-Il17a^{-/-}/f^{-/-} Mice Mice.

The proliferation response and cell death in the polyp environment were compared by pH 3 stain by immunostaining and the number of apoptotic cells by TUNEL using mouse tissue sections. The results suggested no difference in the number of apoptotic cells (FIG. 15), and the number of proliferating cells was understood to be significantly decreased in Apc^{Min/+}-Il17a^{-/-}/f^{-/-} mice (FIG. 16e). Therefore, as a result of CD31 (blood vessel marker) staining, blood vessels were understood to be decreased in the polyp locale of Apc^{Min/+}-Il17a^{-/-}/f^{-/-} mice (FIG. 17).

7. Comparison of Apc^{Min/+}-Il17a^{+/-}/f^{+/-} Mice and Apc^{Min/+}-Il17a^{-/-}/f^{-/-} Mice (6 Months Old)

As a result of comparing the Apc^{Min/+}-Il17a^{+/-}/f^{+/-} mice and Apc^{Min/+}-Il17a^{-/-}/f^{-/-} mice, polyp formation was understood to be significantly decreased in Apc^{Min/+}-Il17a^{-/-}/f^{-/-} mice (FIG. 18). Polyp development was also understood to be significantly decreased in Apc^{Min/+}-Il17a^{-/-}/f^{-/-} mice in comparison to the respective single knockout mice, Apc^{Min/+}-Il17a^{-/-}/f^{+/-} mice and Apc^{Min/+}-Il17a^{+/-}/f^{-/-} mice (FIGS. 18b, c, e, and f).

Based on the above results, definite suppression of polyp formation was seen in Il17a and Il17f single knockout mice, and moreover IL-17F had a greater contribution than IL-17A. Since polyp formation was significantly suppressed in Il17a/f double knockout mice in comparison to the single knockout mice, anti-IL-17F antibody administration or the joint use of anti-IL-17A and -IL-17F antibodies is considered to be effective.

### Example 2

### Suppression of Colorectal Cancer by Anti-IL-17F Antibody and Anti-IL-17A Antibody

(1) Production of Anti-IL-17F Antibody and Anti-IL-17A Antibody

Il17f^{-/-} mice and Il17a^{-/-} mice were immunized with recombinant IL-17F and IL-17A, respectively, to produce anti-IL-17F antibody and anti-IL-17A antibody.

Specifically, Il17f^{-/-} mice and Il17a^{-/-} mice were used as immune animals produced in accordance with the documents described above in Example 1. Commercial (made by R&D Systems Co., Ltd.) recombinant mouse IL-17F and IL-17A were used as antigens. Adjuvant (complete adjuvant (FREUND); RM606-1 made by Mitsubishi Chemical Yatron Co., Ltd.) and 1 mg/mL antigen solution were mixed and emulsified, and the mice were immunized. Immunization was conducted a total of three times, and cell fusion was carried out by PEG. The medium was changed every three days during fusion and inoculation. Culture supernatant from the 96-well plates was sampled at the stage (after 2-3 weeks) when hybridoma colony formation was confirmed, and the following primary screening was performed.

Primary screening was performed by ELISA. First, antigen (recombinant mouse IL-17F or IL-17A) was diluted to 1 μg/mL by PBS, then dispensed in a quantity of 50 μL/well into sensitizing plates (made by NUNC Inc.; Cat No 468667) and allowed to stand overnight at 4° C. The antigen solution was removed thereafter, and 100 μL/well of blocking buffer was dispensed and allowed to stand overnight at 4° C. A quantity of 50 μL/well of the above sampled culture supernatant was added and reacted for 60 minutes at room temperature. After washing three times by 0.05% Tween 20 in PBS, 50 μL/well of goat anti-mouse IgG-POD label (made by MBL Co.; Code. 330) diluted 10,000-fold by dilute buffer (made by MBL Co.) was added and reacted for 60 minutes at room temperature. After washing three times with 0.05% Tween 20 in PBS, 50 μL/well of coloring solution was added, and coloring was induced for five minutes. The reaction was then stopped by adding 50 μL/well of 1.5 mol/L phosphoric acid. After the reaction had stopped, the absorbance was measured at a measurement wavelength of 450 nm and a reference wavelength of 620 nm.

The hybridomas selected based on the culture supernatants judged to be positive by the above primary screen were subjected to a monocloning procedure by the limiting dilution method. Specifically, hybridomas in good condition that had entered the logarithmic growth phase were collected after pipetting with a Pasteur pipette, diluted by medium, and inoculated into 96-well plates by varying the cell concentration so that the cell count per well was from 1 to 32,000 cells. The culture supernatant was sampled from the 96-well plates at the stage (after 1-2 weeks) when formation of hybridoma single colonies had been confirmed.

Next, confirmation of monocloning (isotype confirmation) was performed using an isotyping kit (IsoStrip Mouse Monoclonal Antibody Isotyping Kit; made by Roche Inc., Cat. No. 1-493-027). Specifically, the culture supernatant sampled above that had been diluted 100-fold by PBS was added dropwise to a development tube, and the colored latex beads were resuspended. An isotype strip from the above kit was immersed in the tube, and the bands detected at the specific subclass locations were confirmed every five minutes. These monocloned hybridomas were subcultured from one well of the 96-well plate to a 48-well plate, 24-well plate, and 12-well plate by the limiting dilution method. The cells of one well were recovered by centrifugation, suspended in 500 μL of Cellbanker, placed in a stock tube, and stored at −80° C.

(2) Selection of Neutralizing Antibodies to Mouse IL-17F and IL-17A

The neutralizing activity (in vitro) of the anti-IL-17F antibody and anti-IL-17A antibody screened as described above for mouse IL-17 and FIL-17A was evaluated taking as the indicator (inhibitory activity when ⅓ the amount of hybridoma culture supernatant was added) the induction of IL-6 production when mouse embryonic fibroblasts (MEF) were stimulated (24 hrs) by recombinant IL-17A or IL-17F (R&D Systems Co., Ltd.).

Specifically, mouse embryonic fibroblasts (MEF) were prepared as follows. First, male and female C57BL/6J mice that had reached sexual maturity were housed together, and the presence of a vaginal plug (plug) was confirmed the next morning. The morning of the day on which confirmation was possible was counted as day 0.5. The pregnant mice were laparotomized on day 14.5, and the embryos were removed. The heads and organs of the embryos were removed in cold PBC, and the remainder was minced by scissors. Warming and stirring were carried out for 20 minutes thereafter in 0.05% trypsin solution in a 37° C. incubator. An equal amount of feeder medium (DMEM with nonessential amino acids/sodium pyruvate added, 10% FCS, 100 U/mL penicillin, 100 μg/mL streptomycin) was added to the trypsin solution. After inactivating the trypsin, the solution was passed through a nylon mesh and centrifuged for five minutes at 1000 rpm. The supernatant was discarded, and the cells were suspended in an appropriate amount of feeder medium. Gelatin-coated 15 cm dishes were inoculated with 1×10⁷ cells and cultured in a 37° C. CO₂ incubator. The cells were subcultured the next day or the day after after they had proliferated adequately and were stored frozen after being made to proliferate further.

Next, the in vitro neutralizing activity of the hybridoma supernatant selected by the above primary screening was measured as follows.

MEF prepared as described above were inoculated to make 1-2×10⁴ cells/well (500 μL feeder medium) in 48-well plates and cultured for one day in a 37° C. CO₂ incubator. After removing the medium, 100 μL of fresh medium, 100 μL of hybridoma culture supernatant, and 100 μL of medium containing recombinant (r)IL-17A or rIL-17F (made by R&D Systems Co., Ltd.) were added in that order to the cultured MEF. The rIL-17F was made into a dilution series having final concentrations in the 1.0-50 ng/mL range. rIL-17A was also made into a dilution series having final concentrations in the 0.2-10 ng/mL range. After culturing for 24 hours in a 37° C. CO₂ incubator, the culture supernatant was recovered, and the concentration of IL-6 contained in the culture supernatant was measured by ELISA [using DuoSet (registered trademark): made by R&D Systems Co., Ltd.].

Suitable neutralizing antibodies were selected by again monocloning hybridomas judged to be positive for neutralizing activity by the above measurement and screening them taking as the indicator the inhibition of induction of IL-6 production in the same way as described above. FIGS. 19 and 20 show the inhibitory activity on induction of IL-6 production of several of the selected neutralizing antibodies to IL-17F and IL-17A.

Clone K13-4 (anti-IL-17F antibody) and clones K15-2 and K33-4 (anti-IL-17A antibodies) among the antibodies selected as described above were cultured in serum-free medium (BC Cell™ MAb serum-free medium), and purified antibody (purified by a HiTrap Protein G HP column) was prepared from the supernatant. Specifically, adaptation to serum-free medium was intended by first culturing the hybridoma in serum-containing medium (RPMI 1640, 15% FCS, 100 U/mL penicillin, 100 μg/mL streptomycin), then adding serum-free medium [BD Cell (registered trademark) MAb Serum-Free Medium, 2 mM L-glutamine, 100 U/mL penicillin, 100 μg/mL streptomycin] subsequently to the serum-containing medium at the time of subculture. When growth by culture in 100% serum-free medium had become possible, 3×10⁷ cells were cultured by a dedicated cell tank CELLine (registered trademark) CL-1000 (made by BD Inc.). The hybridoma culture supernatant (up to 15 mL) was recovered once a week. After adding a ¼ quantity of Cleanascite (registered trademark) (Biotech Support Group, LLC) to the recovered culture supernatant and shaking gently for 10 minutes at room temperature, centrifugation was carried out at 2000 rpm, and the supernatant was recovered. After filtering by 0.45 μm filter, purification was conducted by a HiTrap Protein G HP column (made by GE Co.). The concentrated antibody solution eluted by 0.1M glycine-HCl (pH 2.7) was dialyzed (1 hour×2, overnight×1 in a 100-fold quantity of PBS) by Slide-A-Lyzer (registered trademark) Dialysis Cassettes (made by PIERCE) and substituted with PBS. After filter sterilizing by 0.22 μm filter, the protein concentration was determined using BCA Protein Assay (made by PEIRCE). The degree of purification was also confirmed by SDS-PAGE.

The results obtained by reevaluating the neutralizing activity of clone K13-4 (anti-IL-17F antibody) and clones K15-2 and K33-4 (anti-IL-17A antibodies) taking inhibition of induction of IL-6 production as the indicator as above are shown in FIGS. 21 and 22, respectively. Furthermore, clone K15-2 was used as the IL-17A neutralizing antibody in the studies below.

(2) Evaluation of In Vivo Neutralizing Activity (Inhibitory Effect on Intestinal Polyp Formation)

The following antibodies were intraperitoneally administered once a week for a total of six times to four-month-old Apc^{Min/+} mice (C57BL/6J background). The intestine was removed one week after the final administration, and the number of polyps was measured.
Control: 0.5 mg of mouse IgG
Anti-mouse IL-17A antibody (K15-2): first two doses 0.4 mg; 0.2 mg thereafter
Anti-mouse IL-17F antibody (K13-4): 0.2 mg
Both anti-mouse IL-17A antibody and anti-mouse IL-17F antibody

As shown in FIG. 23, the number of polyps 3 mm or larger decreased in mice administered anti-IL-17F antibody in comparison to the control mice. A similar trend was also seen when anti-IL-17A antibody was administered. A slightly greater decrease in the number of polyps than when each was administered individually was seen when anti-IL-17F antibody and anti-IL-17A antibody were administered in combination.

### INDUSTRIAL APPLICABILITY

The inventors have clarified here that inflammatory cytokine IL-1 family molecules and IL-17 family molecules act to promote tumorigenesis during the onset of colorectal cancer and that tumorigenesis can be suppressed by suppressing these cytokines. Based on these results, antibody therapy targeting IL-1 family molecules and IL-17 family molecules, especially IL-17F, can be expected to be newly added as a fifth treatment method, following surgical treatment, chemotherapy, radiation therapy, and immunotherapy as treatment methods for cancer. Therefore, the pharmaceutical composition for treatment of intestinal disease containing an IL-17F inhibitor of the present invention can be utilized in pharmaceutical manufacturing and other such fields.

1. A pharmaceutical composition for treatment of intestinal disease containing an IL-17F inhibitor.

2. The pharmaceutical composition for treatment of intestinal disease according to claim 1 wherein the IL-17F inhibitor is an anti-IL-17F antibody.

3. The pharmaceutical composition for treatment of intestinal disease according to claim 1 wherein an IL-17A inhibitor is used in combination.

4. The pharmaceutical composition for treatment of intestinal disease according to claim 3 wherein the IL-17A inhibitor is an anti-IL-17A antibody and the IL-17F inhibitor is an anti-IL-17F antibody.

5. The pharmaceutical composition for treatment of intestinal disease according to claim 1 wherein the intestinal disease is a polyp or cancer in the intestine.

6. The pharmaceutical composition for treatment of intestinal disease according to claim 5 wherein the polyp or cancer in the intestine is a colonic polyp or colorectal cancer.

7. A method for treating intestinal disease comprising administering a therapeutically effective amount of an IL-17F inhibitor to a patient in need thereof.

8. The method according to claim 7 wherein the IL-17F inhibitor is an anti-IL-17F antibody.

9. The method according to claim 7 wherein a therapeutically effective amount of an IL-17F inhibitor is administered in combination with a therapeutically effective amount of an IL-17A inhibitor.

10. The method according to claim 9 wherein the IL-17A inhibitor is an anti-IL-17A antibody and the IL-17F inhibitor is an anti-IL-17F antibody.

11. The method according to claim 7 wherein the intestinal disease is a polyp or cancer in the intestine.

12. The method according to claim 11 wherein the polyp or cancer in the intestine is a colonic polyp or colorectal cancer.

13. The method according to claim 7 wherein the intestinal disease includes polyps in the intestine of the patient.

14. The method according to claim 8 wherein the intestinal disease includes polyps in the intestine of the patient.

15. The method according to claim 10 wherein the intestinal disease includes polyps in the intestine of the patient.

16. The method of claim 13 further comprising the subsequent step of detecting in the patient a resultant reduction in polyp number.

17. The method of claim 14 further comprising the subsequent step of detecting in the patient a resultant reduction in polyp number.

18. The method of claim 15 further comprising the subsequent step of detecting in the patient a resultant reduction in polyp number.

19. The method of claim 7 wherein the IL-17A inhibitor is an IL-17F-specific siRNA or antisense RNA.

20. The method of claim 7 wherein the IL-17A inhibitor is an IL-17F receptor-specific siRNA or antisense RNA.
